(19) United States
(12) Patent Application Publication
(10) Pub. No.: US 2026/0126371 A1
(43) Pub. Date: May 7, 2026

(54) MULTIMODAL ANALYTICAL SYSTEM FOR ANALYZING BIOLOGICAL FLUIDS

(71) Applicant: General Fluidics Corporation, Watertown, MA (US)

(72) Inventors: Robert Granier, Boston, MA (US); Daniel Morris Hartmann, Arlington, MA (US); Jesse Newton Jones, IV, Woburn, MA (US); Anthony Lawrence Maida, Brighton, MA (US)

(21) Appl. No.: 19/383,175

(22) Filed: Nov. 7, 2025

Related U.S. Application Data
(60) Provisional application No. 63/717,852, filed on Nov. 7, 2024.

(51) Int. Cl. G01N 15/1434; G01N 15/01; G01N 15/10; G01N 15/14; G01N 33/543

(52) U.S. Cl. CPC G01N 15/1434; G01N 15/01; G01N 33/54326; G01N 33/54373; G01N 2015/1006; G01N 2015/1486

(57) ABSTRACT

A modular, point-of-care diagnostic system for analyzing blood is provided comprising a blood sample preparation module and one or more analytical modules including a hematology analysis module and a chemistry analysis module disposed within a housing. The system comprises common biological fluid handling hardware disposed in the housing and configured to convey blood samples or portions thereof between the blood sample preparation module, the one or more analytical modules, and a consumable reagent cartridge comprising one or more reagents for a first set of tests or controls. The system comprises common wash fluid handling hardware for preparing the common fluid handling hardware and fluid-contacting surfaces of the one or more analytical modules for interaction with a second consumable reagent cartridge containing reagents for a second set of tests or controls by the one or more analytical modules.

### CROSS-REFERENCE TO RELATED APPLICATION(S)

This patent application claims the benefit of priority to U.S. Provisional Application No. 63/717,852, filed on Nov. 7, 2024, the entireties of which is incorporated herein by reference.

### TECHNICAL FIELD

Various embodiments of the present disclosure relate generally to systems and methods for a multimodal analytical system and, more particularly, to systems and methods for a multimodal analytical system for analyzing biological fluids.

### BACKGROUND

In vitro diagnostic tests are used throughout healthcare systems to identify, treat and monitor illness. In the majority of these clinical cases, a variety of tests are ordered which may include clinical chemistry, hematology and immunoassay tests. Currently, to perform these tests, multiple systems are required. For example, to perform a complete blood count (CBC), a hematology analyzer is required; to perform comprehensive metabolic panel (CMP), a chemistry analyzer is required; and to perform a cardiac panel, an immunoassay analyzer is required.

In the case of very high-throughput equipment, chemistry and immunoassay testing has been integrated into a single instrument. However, these high-throughput systems are typically large, expensive, and designed for central laboratory environments with high sample volumes. They use bulk reagents for assays making the cost of ownership high, and require dedicated, trained staff to operate. Such systems are not suitable for point-of-care applications where space, cost, and operational complexity constraints exist.

Existing point-of-care in vitro diagnostic products only offer a single testing modality, requiring users to operate multiple instruments to generate information used to support a clinical treatment decision. This approach results in increased operational complexity, higher costs, and longer turnaround times. Healthcare providers must maintain multiple instruments, each requiring separate training, maintenance, and quality control procedures. Similarly, for each instrument, providers must manage separate purchasing, inventory, and waste-stream management, resulting in additional overhead effort and expense.

The separation of testing modalities also creates workflow inefficiencies during testing. Blood samples must be divided and processed through different instruments sequentially or in parallel. This process increases the risk of sample handling errors and extends the total time required to obtain comprehensive diagnostic results.

Finally, because existing point-of-care instruments offer only single modalities, there is no way for any one instrument to provide a synergistic analysis of the multiple data sets being generated. For instance, many disease states require the results from both a chemistry and a hematology analyzer for an accurate diagnosis; for these diseases, the burden of combining and analyzing the results from both instruments falls exclusively on the healthcare provider; the instrument cannot provide a comprehensive dataset.

To address these deficits, a number of multimodal point-of-care instruments are under development. These instruments typically make use of microfluidic, or “lab-on-chip” consumables that combine reagent storage, sample processing, and assay read-out on a disposable chip. These lab-on-chip consumables are not cleaned; waste products from the assays stay in the reaction chamber(s), and the entire consumable is disposed of. Some of these systems do use pipettors to aliquot the sample and transport it to the lab-on-chip disposable, but most of the assay processing is on the disposable cartridge.

Although this approach may provide an attractively simple workflow, it comes with many limitations. First, lab-on-chip consumables tend to be costly. The reaction chambers and fluid channels that enable their function require post-molding assembly processes, such as inserting desiccated reagents, adding filters, and bonding covers. In some cases, the lab-on-chip consumables may also require assembly of additional components such as trocars, pipette tips, and read-chambers. These assembly operations add to the cost and complexity of the consumable. Second, the integration of tests into a microfluidic format often results in an unavoidable coupling of certain processing steps. For instance, some point-of-care devices use disc-shaped consumables which are spun in order to drive reagent transfers and mixing. Since every assay on the consumable is subjected to the same spin-operations at the same times, the process-flow for each assay must be tolerant of these bulk processing steps. This introduces unnecessary complexity to the workflow and may result in inefficiencies in process timing. The complexity of the consumables and the coupling of certain process flow steps drives a third limitation of such systems, which is a lack of flexibility. Adding a new assay to a lab-on-chip system may require a brand-new consumable with new molded features. It may further require that the assay be designed to have a process flow compatible with existing assays on the consumable. Together, these limitations may result in design trade-offs that limit the accuracy and reproducibility of the results. There is, therefore, a need for systems and methods integrating multiple-testing modalities into a single integrated platform to reduce operational costs and complexity, improve user interaction, reduce time-to-results, and enable a broad menu of blood diagnostic tests that may be synergistically analyzed.

There is a further need for a multimodal point-of-care instrument that can deliver central-lab-quality results using low-cost consumables that maintain independent processing-steps for each assay so as to keep the flexibility and quality of the testing high.

The present disclosure is directed to overcoming one or more of these above-referenced challenges.

### SUMMARY

According to an aspect of the present disclosure, an analytical system for analyzing fluids is provided. The system comprises a fluid sample staging module disposed within a housing and configured to stage fluid samples for one or more analyses. The system comprises one or more analytical modules disposed within the housing and comprising one or more of: a cytometry analysis module configured to perform particulate counts; a chemistry analysis module configured to perform chemistry assays; and an immunoassay analysis module configured to perform immunoassay testing. The system comprises common fluid handling hardware disposed in the housing and configured to convey fluid samples or portions thereof between the fluid sample staging module, the one or more analytical modules, and a consumable reagent cartridge inserted into the housing. The consumable reagent cartridge comprises an array of wells accessible to the fluid handling hardware, the array of wells containing reagents for a first set of tests or controls by the one or more analytical modules. The system comprises common waste fluid and wash fluid handling hardware disposed in the housing and in fluid communication with the one or more analytical modules and the common fluid handling hardware, for cleaning the common fluid handling hardware and fluid-contacting surfaces of the one or more analytical modules before interaction with a second consumable reagent cartridge containing reagents for a second set of tests or controls by the one or more analytical modules.

According to other aspects of the present disclosure, the analytical system may include one or more of the following features. The common waste fluid and wash fluid handling hardware may be configured to clean the fluid handling hardware and fluid-contacting hardware of the one or more analytical modules between each set of tests by the one or more analytical modules for reuse of the fluid handling hardware with second and subsequent consumable reagent cartridges, each of the second and subsequent consumable reagent cartridges containing only enough reagents to operate respective second and subsequent set of tests or controls by the one or more analytical modules. The one or more analytical modules disposed within the housing may comprise two or more of: a cytometry analysis module configured to perform complete blood count analysis; a chemistry analysis module configured to perform clinical chemistry assays; and an immunoassay analysis module configured to perform immunoassay testing. The fluid sample staging module may be a plasma-preparation module configured to separate plasma or serum from whole blood samples. The one or more analytical modules may comprise a hematology analysis module configured to perform a complete blood count with 5-part differential, and a chemistry analysis module configured to perform one or more multiplexed panels of clinical chemistry assays, including a comprehensive metabolic panel (CMP). The one or more analytical modules may comprise a hematology analysis module that uses dual-angle Mie-scattering to measure the mean corpuscular hemoglobin concentration (MCHC), mean corpuscular volume (MCV), and mean platelet volume (MPV). The one or more analytical modules may comprise a cytometry analysis module comprising a flow cytometer. The one or more analytical modules may comprise a chemistry analysis module comprising a photometric module that determines the concentration of analytes through fluorescence and/or absorbance measurements through a fluid sample. The one or more analytical modules may comprise an immunoassay analysis module that performs magnetic-bead based chemiluminescent tests. The common fluid handling hardware may comprise one or more probes configured to aspirate and transfer fluids and/or reagents from the consumable reagent cartridge to the one or more analytical modules. The common fluid handling hardware may comprise at least two automated pipettors configured to operate independently, each of the two automated pipettors being mounted to a separate movable arm and/or gantry robot configured to move in two or three dimensions. The analytical system may further comprise a control system disposed within the housing and configured to control the common fluid handling hardware and common waste fluid and wash fluid handling hardware to enable simultaneously or nearly simultaneously analyzing a single fluid sample using two more of the analytical modules. The fluid handling hardware may comprise one or more independently controllable pipette probes configured to: aspirate liquids from the consumable reagent cartridge; and transfer the aspirated liquids to vessels within the one or more analytical modules. The consumable reagent cartridge may be made from a monolithic piece of molded or thermoformed plastic; and may have one or more wells filled with one or more reagents useable by the one or more analytical modules; and wherein each well in the consumable reagent cartridge may be independent and fluidically isolated from every other well in the consumable reagent cartridge, and wherein the consumable reagent cartridge may be sealed with a pierceable foil seal covering its array of wells, making the array of wells and reagents therein accessible to the fluid handling hardware.

According to another aspect of the present disclosure, a modular, point-of-care diagnostic system for analyzing blood is provided. The system comprises a blood sample preparation module disposed within a housing and configured to separate plasma or serum from whole blood samples. The system comprises one or more analytical modules disposed within the housing and comprising one or more of: a hematology analysis module configured to perform particulate counts and perform a complete blood count analysis; and a chemistry analysis module configured to perform chemistry assays. The system comprises common biological fluid handling hardware disposed in the housing and configured to convey blood samples or portions thereof between the blood sample preparation module, the one or more analytical modules, and a consumable reagent cartridge inserted into the housing. The consumable reagent cartridge comprises one or more reagents for a first set of tests or controls, the one or more reagents being accessible to the common biological fluid handling hardware for transfer to the one or more analytical modules. The system comprises common wash fluid handling hardware disposed in fluid communication with the one or more analytical modules and the common biological fluid handling hardware for preparing the common fluid handling hardware and fluid-contacting surfaces of the one or more analytical modules for interaction with a second consumable reagent cartridge containing reagents for a second set of tests or controls by the one or more analytical modules.

According to another aspect of the present disclosure, a modular, point-of-care diagnostic system for analyzing blood is provided. The system comprises a blood sample preparation module disposed within a housing and configured to separate plasma or serum from whole blood samples. The system comprises one or more analytical modules disposed within the housing, the one or more analytical modules comprising: a hematology analysis module configured to perform particulate counts and perform a complete blood count analysis; and a chemistry analysis module configured to perform chemistry assays.

According to another aspect of the present disclosure, a modular analytical system for analyzing fluids is provided. The system comprises a fluid sample staging module disposed within a housing and configured to stage fluid samples for one or more analyses. The system comprises two or more analytical modules disposed within the housing and comprising one or more of: a cytometry analysis module configured to perform particulate counts; a chemistry analysis module configured to perform chemistry assays; and an immunoassay analysis module configured to perform immunoassay testing. The system comprises common fluid handling hardware disposed in the housing and configured to convey fluid samples or portions thereof between the fluid sample staging module, the one or more analytical modules, and a consumable reagent cartridge inserted into the housing, the consumable reagent cartridge comprising an array of wells accessible to the fluid handling hardware. The system comprises common waste fluid and wash fluid handling hardware disposed in the housing and in communication with the two or more analytical modules and the common fluid handling hardware. The system comprises a controller configured to control the common fluid handling hardware to simultaneously interact with the two or more analytical modules, to generate a diagnostic report of a fluid sample based on outputs received from the two or more analytical modules, and to control the common wash fluid handling hardware to clean the common fluid handling hardware before analysis of a second fluid sample.

According to other aspects of the present disclosure, the modular analytical system may include one or more of the following features. The controller may be further configured to control the common fluid handling hardware to convey portions of a fluid sample from the fluid sample staging module to wells of the consumable reagent cartridge, and to simultaneously convey fluid from two or more wells of the consumable reagent cartridge to vessels of the two or more analytical modules. The controller may be further configured to control the common waste fluid and wash fluid handling hardware to clean the common fluid handling hardware and fluid-contacting surfaces of the two or more analytical modules between analyses of second and subsequent fluid samples using second and subsequent consumable reagent cartridges. The controller may be further configured to: use outputs from the two or more analytical modules to identify correlations between the outputs, organize the outputs relative to a particular diagnosis or set of diagnoses, or initiate or recommend additional analysis based on the outputs; and control the movement of fluid between the fluid sample staging module, wells of the consumable reagent cartridge, vessels of the two or more analytical modules to minimize time to produce the outputs from the two or more analytical modules.

Aspects of the present disclosure include a multimodal analytical system that adapts multiple technologies used by large laboratories and seamlessly integrates them into a single, compact analyzer that is easy to operate. The method of integrating these technologies as described by the present disclosure reduces operational costs and complexity, improves user interaction (ease-of-use, quality and cost), reduces time-to-results, and enables a broad menu of blood diagnostic tests that can be analyzed synergistically.

The present disclosure describes hardware used to perform simultaneous analysis of one or more samples using various analytical methods (modalities) to provide healthcare providers and patients with complete or broader diagnostic information used to screen for and/or diagnose illnesses.

The multimodal analytical system comprises at least two primary subsystems, at least three primary subsystems, or at least four primary subsystems for simultaneous analysis of one or more samples. In one embodiment, the multimodal system comprises four primary subsystems including:

A blood fractionation system to separate plasma or serum from whole blood. This could be a centrifuge or another technology that is integrated into the multimodal analytical system or sample collection device.

An analytical module(s) for performing multiplexed panels of clinical chemistry assays. An example of such a module that can be integrated into the multimodal analytical system of the present disclosure is the integrated photometry module (IPM), which is described in PCT Application No.: PCT/US2024/028033, which is hereby incorporated by reference in its entirety.

An analytical module for performing multiplexed immunoassay panels. An example of such a module that can be integrated into the multimodal analytical system of the present disclosure is the integrated immunoassay module (IAM) described in U.S. Provisional Application No.: 63/717,779, which is hereby incorporated by reference in its entirety.

An analytical module for performing complete blood counts (CBC's) An example of such a module that can be integrated into the multimodal analytical system of the present disclosure is the integrated cytometry module (ICM) described in US Provisional Application No.: 63/717,782 which is hereby incorporated by reference in its entirety.

These four sub-systems are integrated through a common backbone of the multimodal analytical system. In some embodiments, the backbone of the analytical system includes a housing, a motion control system, a pipetting system for aliquoting and mixing fluids, a cleaning system, waste-removal system, electronics, power, and control software. In addition, the disposable reagent cartridge(s) and sample vessels used by the system may be shared by some or all the subsystems.

A further aspect of the present disclosure includes one or more single-use consumable(s) for use with the multimodal analytical instrument. When in use, the consumables consist of simple arrays of open wells containing the reagents necessary to run the desired tests on a sample. The consumables have substantially identical wells, so that the locations for the reagents on the consumable(s) are not coupled to any physical aspects of the consumables. Some wells on the consumables may serve as locations for sample processing (mixing or heating, for example), but reagents and sample may also be transferred by the system hardware (such as the common motion control system, pumps, and pipettors) directly to vessels within the modules for processing in the same chambers wherein the assay reactions and read-outs occur.

It is a further aspect of this instrument that the processing steps for each assay are largely independent from those of any other assay. Reagents and sample for each assay are individually aspirated from the consumable and dispensed into reaction chambers within the modules. Thus, for each assay, the system maintains direct, independent control of assay parameters including reagent volume, sample volume, and mixing parameters, along with heating, incubation, and reading times.

It is a further aspect of this disclosure that the simple consumables, the modular nature of the modalities, and the independent control of assay parameters described above ensures that the system maintains flexibility and expandability. For instance, if an additional assay is added to the menu, the consumable does not need to be altered, except that one or two additional wells on the consumable must be filled with reagents for the new assay. The instrument may then be instructed to perform the new assay with parameters uniquely chosen for that assay. The reagent volume, sample volume, mix time, incubation time, and read time used in the new assay may be chosen without impacting any of the existing assays.

An additional aspect of the present disclosure is that the results from the assays performed on the multiple modalities of the instrument may be organized and reported in ways that help the healthcare practitioner analyze the data. For instance, results from different modalities that indicate a particular disease state may be automatically flagged or grouped together. Quantitative correlations between modalities may be reported and may help the practitioner determine a diagnosis from multiple candidates, or may automatically trigger additional testing on the sample.

### BRIEF DESCRIPTION OF THE DRAWINGS

These and other features, aspects, and advantages of the present invention will become better understood with regard to the following description, and accompanying drawings, where:

FIG. 1 depicts a table of exemplary blood diagnostic tests ordered for various clinical presentations, according to one or more embodiments.

FIG. 2A-2B depict two systems for analyzing fluids, according to one or more embodiments.

FIG. 2A depicts workflow using current laboratory equipment involving at least three distinct instruments, and associated sample preparation time, to run separate blood diagnostic tests.

FIG. 2B depicts an integrated multimodal analytical system and associated streamlined workflow, according to one or more embodiments.

FIG. 3 depicts a diagram of an integrated multimodal analytical system and associated workflow showing the common backbone and modules of the multimodal analytical system, according to one or more embodiments.

FIG. 4 depicts a schematic diagram of the various component modules and common fluidic elements of the integrated multimodal analytical system, according to one or more embodiments.

FIGS. 5A-5B depict biological fluid diagnostic test consumables used by the integrated multimodal analytical system of FIGS. 3 and 4, according to one or more embodiments.

FIG. 5A depicts an exemplary set of consumables for the integrated multimodal analytical system, according to one or more embodiments.

FIG. 5B depicts an alternative set of consumables for the integrated multimodal analytical system, according to one or more embodiments.

FIG. 6A depicts a top cross-sectional view of an exemplary layout of the modules of the integrated multimodal analytical system, according to one or more embodiments.

FIG. 6B depicts hatching over fly-zones for two working arms of the integrated multimodal analytical system, the hatching demonstrating low overlap and/or interference between those two fly-zones, according to one or more embodiments.

FIG. 7 depicts a timing diagram for two working arms of the integrated multimodal analytical system, demonstrating high simultaneous productivity of those two working arms, according to the hardware arrangement depicted in FIGS. 6A-6B.

FIG. 8 depicts a top cross-sectional view of another exemplary layout of the modules of the integrated multimodal analytical system, according to one or more embodiments.

FIG. 9 depicts a timing diagram for two working arms of the integrated multimodal analytical system, demonstrating high simultaneous productivity of those two working arms, according to the hardware arrangement depicted in FIG. 8.

FIG. 10 depicts a custom Hemolysis, Icterus, and Lipemia (HIL) circuit board attached to a photometric module of the integrated multimodal analytical system, according to one or more embodiments.

FIG. 11 depicts an absorption spectrum for the H, I, and L interferents, with the LED wavelengths called-out, according to one or more embodiments.

FIG. 12 provides an orthographic interior view of the integrated multimodal analytical system, according to one or more embodiments.

FIG. 13 provides a perspective view of an interior layout of the integrated multimodal analytical system, according to one or more embodiments.

FIGS. 14A-14B depict views of a stir-pipettor of the integrated multimodal analytical system, according to one or more embodiments.

FIG. 14A depicts an isometric view of the stir-pipettor, according to one or more embodiments.

FIG. 14B depicts a bottom view of the stirpipettor with a zoomed-in view of the pipette as it turns, according to one or more embodiments.

FIG. 15 depicts an exemplary waste/wash station of the fluidic system of the integrated multimodal analytical system, according to one or more embodiments.

FIG. 16 depicts a pipettor configuration and orientation in association with an integrated photometry module (IPM), a waste station, and a sample holder of the integrated multimodal analytical system, according to one or more embodiments.

FIG. 17 depicts another conceptual schematic of the various modules of the integrated multimodal analytical system, according to one or more embodiments.

FIG. 18 depicts linearity curves for a Basic Metabolic Panel (BMP) measured from one or more modules (e.g., IPM) of the multimodal analytical system, according to one or more embodiments.

FIG. 19 depicts linearity curves for a Complete Blood Count (CBC) measured from one or more modules (e.g., ICM) of the multimodal analytical system, according to one or more embodiments.

FIG. 20 shows Immunoassay (Troponin hsTni) performance data measured from one or more modules (e.g., integrated immunoassay module IAM) of the integrated multimodal analytical system, according to one or more embodiments.

FIG. 21 depicts a schematic diagram of a computing bus and processing controller of the integrated multimodal analytical system, according to one or more embodiments.

### DETAILED DESCRIPTION OF THE DRAWINGS

The present disclosure describes a multimodal system for analyzing biological fluids, and methods of use thereof. In some embodiments, the system integrates multiple analytical technologies within a single unified platform. The system may be capable of performing clinical chemistry, immunoassay, and hematology tests using a common set of electronics, fluidics, and user interface components. In certain embodiments, the system incorporates a modular arrangement of analytical subsystems, such as modules for blood fractionation, photometry, immunoassay, and cytometry. These modules may be operably linked to shared resources, including automated pipetting, reagent cartridges, and waste removal mechanisms. The system may be designed to receive biological samples and process them through automated workflows that allocate, aliquot, and deliver fluids to selected analytical modules based on the testing requirements.

In some embodiments, the system is operable with a variety of sample types, including but not limited to whole blood, plasma, cerebrospinal fluid, urine, and other dissolved biological materials. The system may be deployed in various clinical, research, or point-of-care environments to enable efficient, high-throughput diagnostics from a broad range of biological specimen types. The integrated configuration may reduce manual handling, minimize error potential, and provide flexibility for a wide menu of diagnostic assays. Furthermore, the integrated configuration enables data analytics to be performed on datasets originating from multiple modalities enabling automated software to detect patterns and correlations that may trigger reflex testing or help providers consider likely diagnoses. Various embodiments and aspects of the multimodal analytical system are described in further detail below and illustrated in the accompanying figures.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value. In this disclosure, unless stated otherwise, any numeric value may include a possible variation of ±10% in the stated value.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

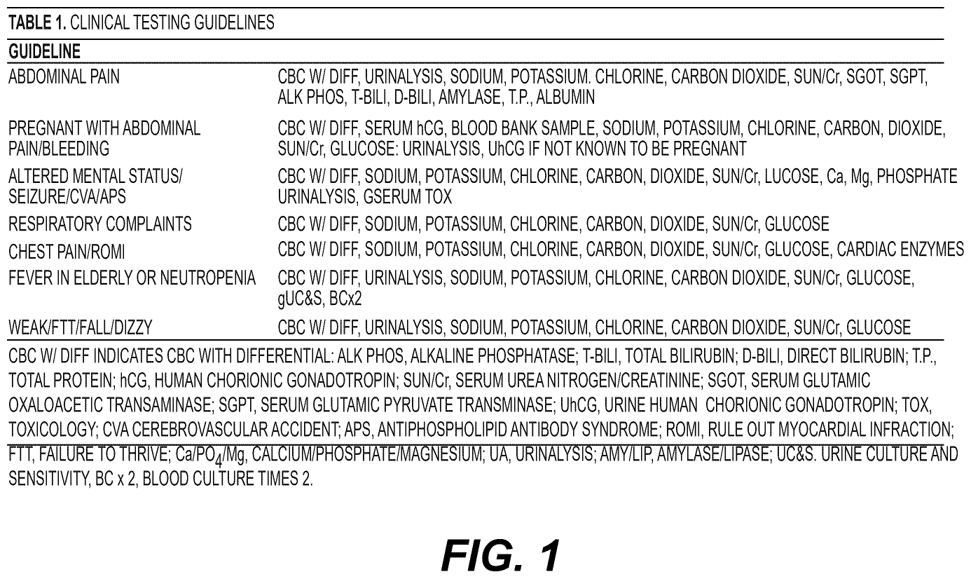

Turning now to the appended figures, FIG. 1 depicts tests ordered for clinical presentations, according to one or more embodiments. In some embodiments, the figure provides a representation of how different types of diagnostic assays, such as clinical chemistry, immunoassay, and hematology tests, may be selected and organized in response to specific patient symptoms or conditions. The depicted workflow may show representative test panels and the correspondence between presenting complaints and relevant laboratory diagnostics. In particular, FIG. 1 depicts exemplary clinical testing guidelines for testing for abdominal pain, pregnancy with abdominal pain and bleeding, altered mental status (including seizure, CVA, APS), respiratory complaints, chest pain (including ROMI), fever in elderly (or neutropenia) persons, weakness (including FTT, fall, or dizzy), etc.

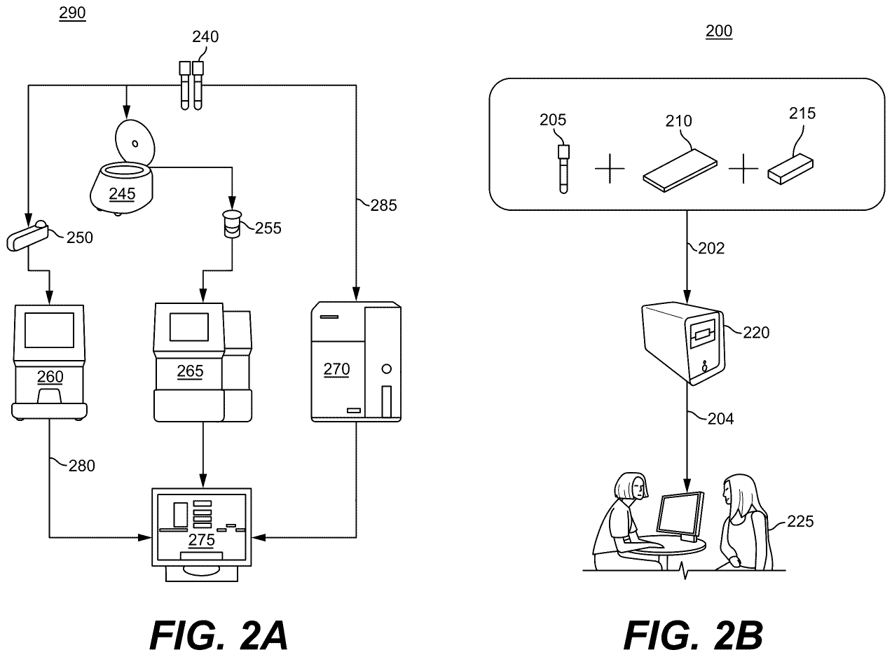

FIG. 2A depicts a typical workflow for using conventional laboratory equipment involving at least three separate instruments to perform in-vitro clinical testing of biological fluids. System 290 includes a discrete analyzer setup in which biological sample(s) 240 are processed through separate preparation staging devices 245, 250, 255, followed by analysis using dedicated immunoassay system 260, chemistry system 265, and hematology system 270. The data output is accessed via terminal 275. The process may require manual or semi-automated sample preparation time of approximately 10 to 15 minutes, and total testing time of approximately 25 to 35 minutes, reflecting the need for multiple handling steps and separate testing platforms.

FIG. 2B depicts an integrated multimodal system workflow, according to one or more embodiments of the present disclosure. System 200 may function as an integrated analytical system designed for a streamlined workflow. System 200 may include sample(s) 205, cartridges 210, and cartridges 215, which are processed by a single integrated multimodal analytical system 220. System 200 may involve sample preparation 202 having a total time of less than or equal to approximately one or two minutes, and a sample testing process 204 having a total time of approximately 15 to 20 minutes, before presenting results to a user 225, such as a clinician (doctor and/or nurse) and/or patient.

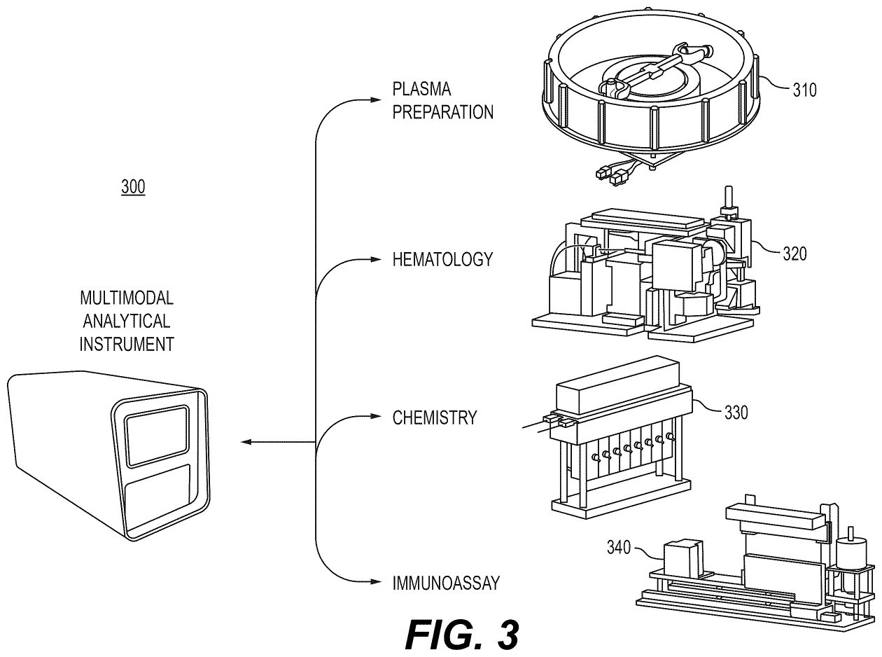

FIG. 3 depicts a diagram showing the common backbone and modules of the multimodal analytical system of FIG. 2B, according to one or more embodiments. As shown in FIG. 3, the integrated multimodal analytical system may be embodied as an integrated, single-housing, multi-module system 300 for analyzing biological fluids. In one embodiment, system 300 may generally include four modules, including a plasma preparation module, or pre-analytical module (PAM) 310, integrated hematology module (ICM) 320, chemistry module, or integrated photometry module (IPM) 330, and immunoassay module (IAM) 340. The system 300 may include a housing to enclose the modules.

In one non-limiting example, the multimodal system comprises four modules comprising:
(1) A blood fractionation system to separate plasma or serum from whole blood. This could be a centrifuge or another technology that is integrated into the multimodal analytical system or sample collection device.
(2) An analytical module(s) for performing multiplexed panels of clinical chemistry assays. An example of a clinical chemistry module that can be integrated into the multimodal analytical system of the present disclosure is the integrated photometry module (IPM) described in PCT Application No.: PCT/US2024/028033, which is hereby incorporated by reference in its entirety.
(3) An analytical module for performing multiplexed immunoassay panels. An immunoassay module (IAM) that can be integrated into the multimodal analytical system of the present disclosure is described in U.S. Provisional Application No.: 63/717,779, which is hereby incorporated by reference in its entirety.
(4) An analytical module for performing complete blood counts (CBC's). An example of such a module that can be integrated into the multimodal analytical system of the present disclosure is the integrated cytometry module (ICM) described in U.S. Provisional Application No.: 63/717,782 which is hereby incorporated by reference in its entirety.

In some embodiments, these four sub-systems are integrated through a common backbone of the multimodal analytical system. In some embodiments, the backbone of the analytical system includes a housing, a motion system, a fluid transfer system, such as pipettors and pumps for aliquoting and mixing fluids, a cleaning system, waste-removal system, electronics, power, and control software. In addition, the disposable reagent cartridge and sample vessels may be shared by some or all of the subsystems.

As shown in FIG. 3, system 300 may include a single, integrated housing, which contains common electronics and controls, pipetting for aliquoting and mixing, a cleaning system, a waste removal system, reservoirs (for clean, system, and waste fluids), reagent cartridge(s) and sample vessels for servicing the various modules 310, 320, 330, 340 of the system 300. Thus, the plasma preparation module 310 may separate plasma or serum from whole blood samples. The hematology module 320 may perform complete blood counts on biological samples. The chemistry module 330 may perform multiplexed panels of clinical chemistry assays. The immunoassay module 340 may measure analytes through immunoassay techniques. The system 300 may further include a common infrastructure for the four analytical modules.

Thus, in some embodiments, the integrated, multimodal analytical system comprises a single housing containing multiple common sub-systems shared by multiple analytical modules. In some embodiments, the multimodal system comprises shared electronics and control systems. In some embodiments, the multimodal system comprises shared means for motion control, pipetting, aliquoting, and mixing. In some embodiments, the multimodal system comprises a shared waste removal system. In some embodiments, the multimodal system comprises shared reservoirs for cleaning, system, and waste fluids. In some embodiments, the multimodal system comprises shared reagent cartridges and sample vessels.

In some embodiments, the multimodal system comprises one or more modules. In one embodiment, the multimodal system comprises two or more modules. In some embodiments, the multimodal system comprises three or more modules. In some embodiments, the multimodal system comprises four or more modules. In some embodiments, the multimodal system comprises five or more modules. In some embodiments, the multimodal system comprises six or more modules. In some embodiments, the multimodal system comprises seven or more modules. In some embodiments, the multimodal system comprises eight or more modules. In some embodiments, the multimodal system comprises nine or more modules. In some embodiments, the multimodal system comprises ten or more modules.

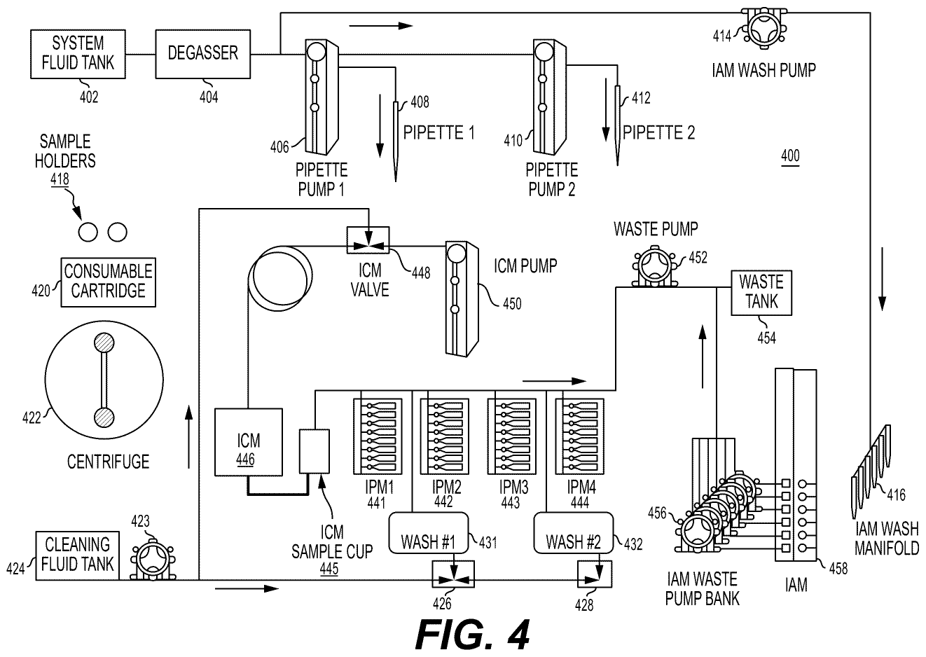

FIG. 4 depicts a schematic diagram of the various component modules of the integrated multimodal analytical system, according to one or more embodiments. At a high level, as shown in FIG. 4, and consistent with the conceptual diagram of FIG. 3. an exemplary system includes the four modules (centrifuge, ICM, IAM, and IPMs) and the supporting system backbone that includes a system fluid tank, degasser, pumps, pipettors, cleaning fluid tank and associated pump, waste and wash stations, and a waste tank. In particular, as shown in FIG. 4, system 400 includes a system fluid tank 402, degasser 404, a first pipette pump 406, a first pipette 408, a second pipette pump 410, a second pipette 412, an IAM wash pump 414, an IAM wash manifold 416, sample holders 418, consumable cartridge 420, centrifuge 422, cleaning fluid tank 424, cleaning fluid pump 423, valve 426, valve 428, waste/wash station 431, waste/wash station 432, first IPM 441, second IPM 442, third IPM 443, fourth IPM 444, ICM sample cup 445, ICM 446, ICM valve 448, ICM pump 450, waste pump 452, waste tank 454, IAM waste pump bank 456, and IAM 458.

The various pumps of the system may be used to remove fluid through an outlet of one or more fluid containers of the module(s). The fluidic pressure difference across the fluid can be formed with the use of, for example, a pump that is appropriately connected to the outlet, of one or more fluid containers of the one or more modules. In FIGS. 4A-4B, one example of this is labeled as the waste pump 452. Alternatively, the fluidic pressure can be formed with the use of a pressure that is applied to the inlet end of the fluid container, resulting in fluid that is pushed out of the outlet. In some embodiments, the pump is a peristaltic pump, a membrane pump, or a diaphragm pump. In some embodiments, the pump comprises an evacuated gas cylinder, configured to create a pressure difference across the fluid in the fluid container by pulling on their outlets. In some embodiments, the pump is a pneumatic pump. In some embodiments, the pump comprises a pressurized gas cylinder to create a pressure difference across the fluid in the container by applying pressure to their inlets.

In some embodiments, such as that shown in FIG. 4 where a waste tank 454 is present, the system comprises a channel or tube connecting the one or more individual fluid containers in the one or more modules to the waste tank. The waste tank can include a common single waste collection reservoir (storage volume) as shown in FIG. 4.

In some embodiments of the multimodal analytical system in which a cleaning fluid tank 424 is present, the cleaning fluid tank may be fluidically connected to a pipettor so that the pipettor can wash itself and dispense cleaning fluid into fluid container(s) to clean them. Alternatively, the cleaning fluid may be plumbed to waste/wash stations such as is shown in FIG. 4. Cleaning solution delivered to the waste/wash stations may be aspirated by the pipette to clean itself and may be delivered by the pipette to other parts of the system that require cleaning with a cleaning solution.

In some embodiments of the multimodal analytical system in which a cleaning fluid pump 423 is present, the cleaning fluid pump can be used to pump cleaning fluid from the cleaning fluid tank, to the ICM flowcell and sample-cup. In some embodiments of the multimodal analytical system in which a cleaning fluid pump 423 is present, the cleaning fluid pump can be used to pump cleaning fluid to IPM cuvettes or IAM reaction vessels, and to waste/wash stations, where it may be used to clean the pipettors.

In some embodiments of the multimodal analytical system in which a degasser 404 is present, the degasser can be used to degas fluids before they are used in the system.

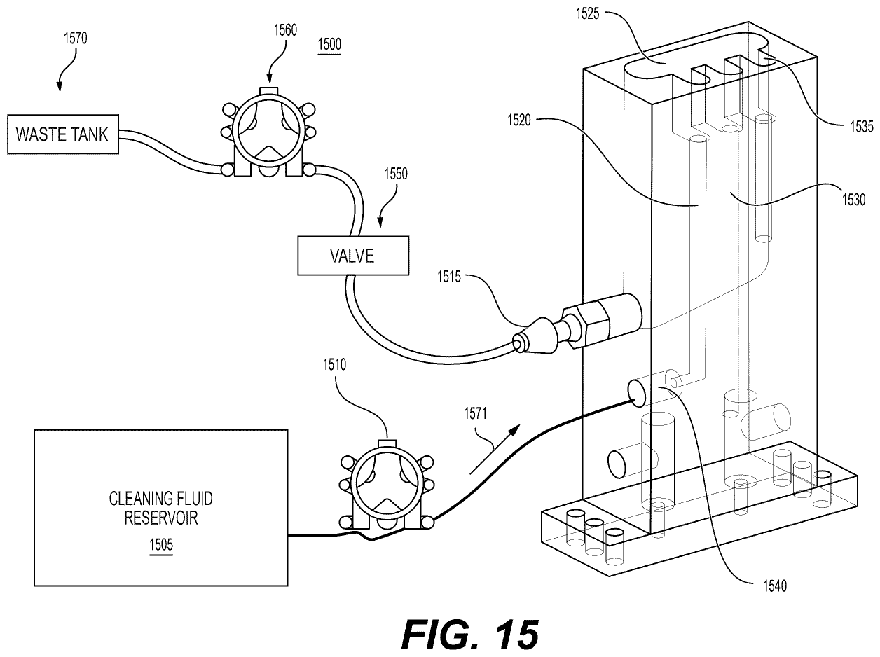

In some embodiments the multimodal analytical system includes at least one waste/wash station. FIG. 4 shows an exemplary system with waste/wash station 431 and waste/wash station 432. These will be described further below with reference to FIG. 15.

In some embodiments the multimodal analytical system includes a system fluid tank 402 and may also include an associated degasser 404. In some embodiments the degasser may not be needed. The system fluid is used to prime fluidic lines to support aspirate and dispense operations, and may further be used for washing operations.

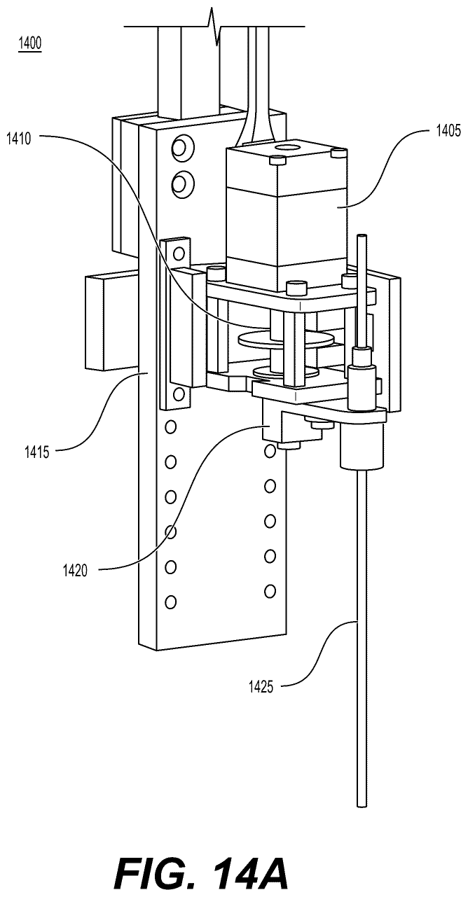

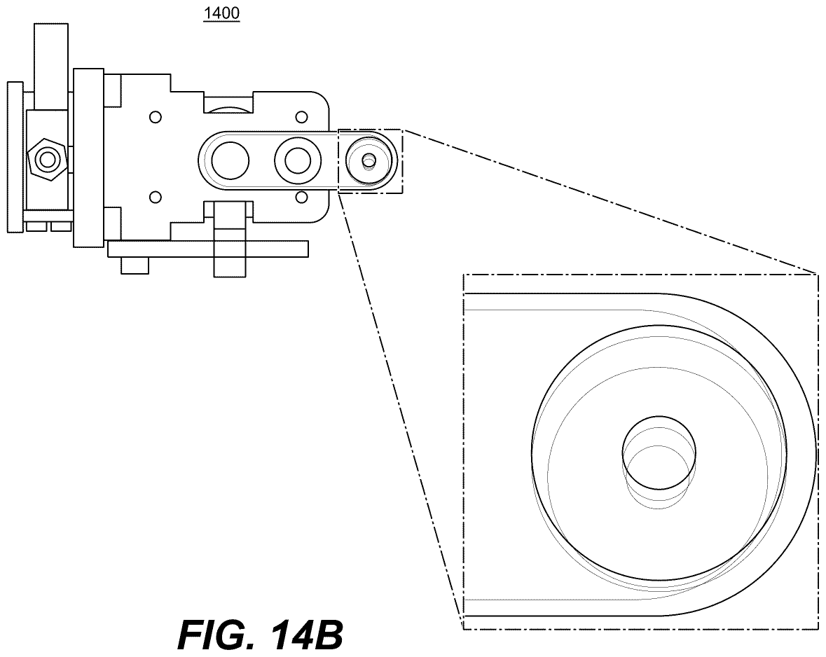

In some embodiments the multimodal analytical system includes at least one pipette with an associated pump and valve that can be used to aspirate and dispense fluids. FIG. 4 shows an exemplary system with two pipettes 408 and 412 and their corresponding pumps 406 and 410. In some embodiments the at least one pipette is mounted to at least one robot arm so that it can be moved to different locations in the instrument. In some embodiments there are two pipettes and each one is mounted to a separate movable arm so that the two arms can be individually controlled and the two pipettes can be moved to different locations within the system at the same time. In some alternative embodiments, more than one pipette is mounted to the same movable arm. In some embodiments the robot arms are gantry robots, each capable of moving linearly in three dimensions. In some embodiments, the robot arms only move in one or two dimensions. First pipette 408 and second pipette 412 and their associated stirring capability will be described further with reference to FIGS. 14A-14B

In some embodiments the multimodal analytical system includes an additional system pump 450. In some embodiments the additional system pump may primarily serve the ICM, and may be referred to as the ICM pump. The ICM pump 450 may be a high-precision pump. Additional system hardware of system 400 may include valves, sample holders, and wash pumps, and these may be components for use in bioinstrumentation.

To process a single biological fluid sample, system 400 may operate by first receiving the sample in a designated sample holder 418, where it is accessed by at least one of the pipettes 408, 412 controlled by corresponding pipette pumps 406, 410. A portion of the sample may be transferred to a consumable cartridge 420 where, for instance, it may be mixed with pre-analytical reagents such as diluents, dyes, or lysing buffers. Another portion of the sample may be transferred to the centrifuge 422 and subjected to separation. For instance, if the sample is whole blood, it may be spun in centrifuge 422 to obtain plasma and cellular fractions. Afterwards, portions of the separated fractions may be transferred to consumable cartridge 420 for pre-analytical processing.

Using pipettes 408 and 412, reagents and sample may be distributed from sample holders 418, reagent cartridge 420, and centrifuge 422 to analytical modules such as the IAM 458 for immunoassays, ICM sample cup 445 for hematology assays, and IPMs 441, 442, 443, and 44 for clinical chemistry assays.

Following analysis, some or all of the modules may make use of system hardware to clean durable components. For instance, IPM1 441, IPM2 442, IPM3 443, IPM4 444, IAM 458, ICM sample cup 445, and ICM 446 may contain reusable components that are washed by priming system fluid into those components using pipettes 408 and 412. Similarly, pipettes 408 and 412 may aspirate cleaning fluid from waste/wash stations 431 and 432 and dispense that cleaning fluid into each of the modules for additional cleaning.

In some embodiments waste fluids are managed through a network including the waste/wash stations 431, 432, waste pump 452, IAM waste pump bank 456 and waste tank 454.

In some embodiments some modules use dedicated hardware. For instance, the IAM may use IAM wash manifold 416, IAM wash pump 414, and IAM wash pump bank 456. The ICM may use dedicated pump 450. However these dedicated hardware may still make use of system-level resources such as the system fluid tank 402, degasser 404, and waste tank 454.

The integrated operation of these components enables automated sample handling, processing, analysis, and cleaning within a single workflow, as illustrated in FIG. 4.

In some embodiments, the backbone of the multimodal analytical system comprises one or more pumps. In some embodiments, the one or more pump(s) is attached to a tube or channel that is connected to a fluidic valve, wherein when the fluidic valve is closed, the pump is fluidically disconnected from a fluid container, such as a cuvette or cup or vessel, and wherein when the valve is open, the pump is fluidically connected to the fluid container. In some embodiments, the fluid container can be a sample cup or a cuvette.

In some embodiments, the multimodal analytical system comprises reusable embedded fluidic components as part of its integrated backbone. In some embodiments, the multimodal analytical system comprises a liquid handling system involved in aliquoting, transporting and mixing of samples and reagents. In some embodiments, the multiple analytical modules are serviced by one or more pipetting systems. In some embodiments, the pipettes of the multimodal analytical system interact directly with containers in the modules where the assays are performed. In some embodiments, the multimodal analytical system enables sample processing (e.g. heating, mixing, incubation) in the modules in the same containers where the assay read-outs are performed. In some embodiments the clinical chemistry and immunoassay containers where assay read-outs occur may be directly addressed by at least one pipettor.

In some embodiments, the multimodal analytical system comprises at least two analytical modules and at least two independent pipettors each configured to access at least one module directly, where both pipettors can also access one or more single-use consumables containing reagents and at least one sample. This allows the pipetting systems to control the following processes directly: sample and reagent aliquoting, analytical modality selection, mixing, and assay start time. All the modules on the system share a common backbone that includes a housing, a pipetting system for aliquoting and mixing fluids and for moving those fluids from one place to another, along with reservoirs for system fluid, cleaning fluid, and waste collection. In addition, all modules share common electronics, power, and control systems, including a common software architecture. Furthermore, the ICM and IPM modules share a common waste pump.

In some embodiments the system backbone includes a motion control system that comprises two or more arms that can move independently. To ensure that the instrument can complete all assays in a short amount of time, the modules are positioned such that each arm takes responsibility for particular assays, wherein the location and timing of each assay run on the modules is specified.

In some embodiments, the multimodal analytical system of the present disclosure is capable of removing waste independently and simultaneously or near-simultaneously from each module. In some embodiments, the multimodal analytical system uses reusable fluidic components which may be cleaned by one or more of the following steps: (a) using the pipettor to fill the fluidic vessels (e.g. cuvettes or sample cup) with system fluid while the corresponding waste valve is closed; (b) optionally mixing the system fluid actively with any residual fluids in the sample vessels (e.g. by stirring with a pipette probe and/or aspirating and dispensing the fluid in the vessel while the waste valve is closed; and (c) opening a valve to connect the vessel to a downstream pump and removing the waste fluid from the fluidic vessel by using the pump to generate a negative pressure that causes fluid to be drawn out of the vessel's outlet. It should be noted that step (b) may not be necessary because some mixing of the system fluid with residual fluids happens by natural diffusion, and by the turbulent mixing inherent in the rapid injection and withdrawal of the liquid into and out of the vessel(s).

As described above with respect to FIG. 3, in some embodiments where the integrated multimodal analytical system of FIG. 4 comprises an integrated ICM, the ICM and components thereof is as described in U.S. Provisional Application No.: 63/717,779, which is hereby incorporated by reference in its entirety. In some embodiments, where the integrated multimodal analytical system of FIG. 4 comprises an integrated IAM, the IAM and components thereof is as described in U.S. Provisional Application No.: 63/717,782, which is hereby incorporated by reference in its entirety. In some embodiments where the integrated multimodal analytical system of FIG. 4 comprises an integrated IPM, the IPM and components thereof is as described in US PCT Application No.: PCT/US2024/028033, which is hereby incorporated by reference in its entirety.

In addition to the integrated modules described in the present disclosure, the system further comprises an interface designed to accept the sample and single-use consumable(s), which are inserted by the user. Upon insertion, the instrument automatically positions the sample and consumable(s) so they can be accessed by the pipetting system.

After placing the sample and consumable(s) on the instrument, the method comprises the user selecting the tests-to-be-run on a GUI. The pipetting system takes an aliquot of the sample and may add it to the blood fractioning system (e.g. the centrifuge module), to create a serum or plasma fraction for assays requiring those factions. While blood fractionation is occurring, whole blood assays (e.g., CBC) may be started using one or more of the system pipettors. Once blood fractionation is complete, the system interweaves hematology, clinical chemistry, and immunoassays between the two or more pipetting systems in a way that may reduce testing time for one or more assays.

For example, in some embodiments, for each assay, a pipette will aliquot the specified volume of a reagent for an assay and transport it to a receptacle in the appropriate analytical module for that assay. In some embodiments, the pipetting system will then collect the appropriate sample volume from the sample container and add it to the receptacle containing the reagent. In some embodiments, the pipettor will then mix the reagent and sample by using the pipette probe to stir the mixture or to aspirate and dispense the mixture one or more times. If the assay only requires a single reagent, in some embodiments, the pipetting system can move on to working on another assay. In some embodiments, if the assay is a two-step assay (2 reagents), the pipetting system can do other tasks (cleaning or another assay) during the incubation period, and return to the first assay when it is ready to proceed.

In some embodiments, the analytical modules in the system are responsible for analyzing the processed samples and quantifying the analytes for each assay. Once the assays are completed, a common waste pump pulls the fluids from each reusable fluid-container and disposes of them via a fluidic network, to a common waste container.

In some embodiments, the reusable fluid-containers in the modules are cleaned between runs by the pipettor, which adds a wash fluid to each of the fluid receptacles. In some embodiments, this fluid is the system fluid. In some embodiments, if multiple cleaning cycles are required, the next cleaning cycle will begin after the module disposes of the cleaning mixture in the same way it disposes of the assay mixtures. Cleaning of the probe occurs in a similar process using one or more wash stations within the system.

In some embodiments, a more intensive maintenance cleaning can also be performed using a dedicated maintenance cartridge. This process is designed to extend the life of the reusable fluidic components of the system and may use strong detergents or other chemicals (versus the chemically inert system fluid used to clean between tests).

Quality control and calibration are performed in a manner similar to that of sample testing. In some embodiments, the quality control and calibration kits are contained in disposable cartridges. In some instances, controls and/or calibrators may be included in the same cartridge/consumable as reagents for the assays so that they can be used during each test. In other cases, controls and/or calibrators may take the place of a sample, and a consumable reagent cartridge may be run independent of sample testing at intervals determined by the user.

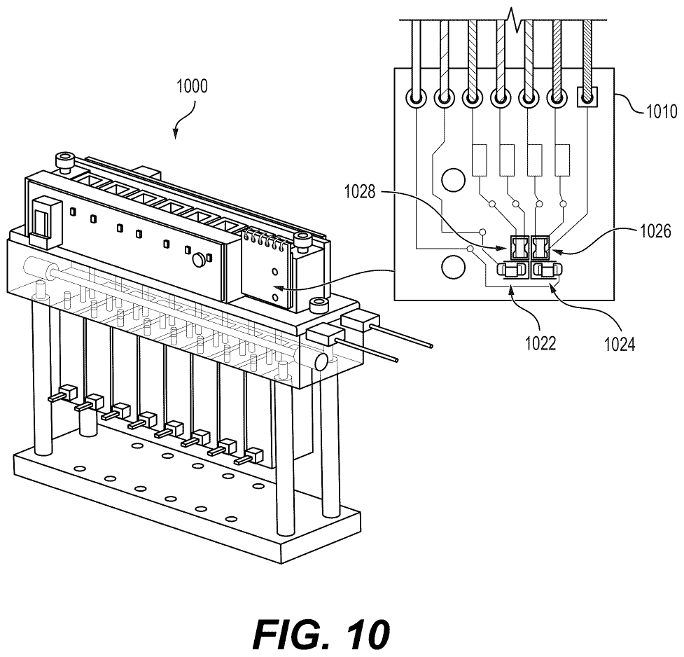

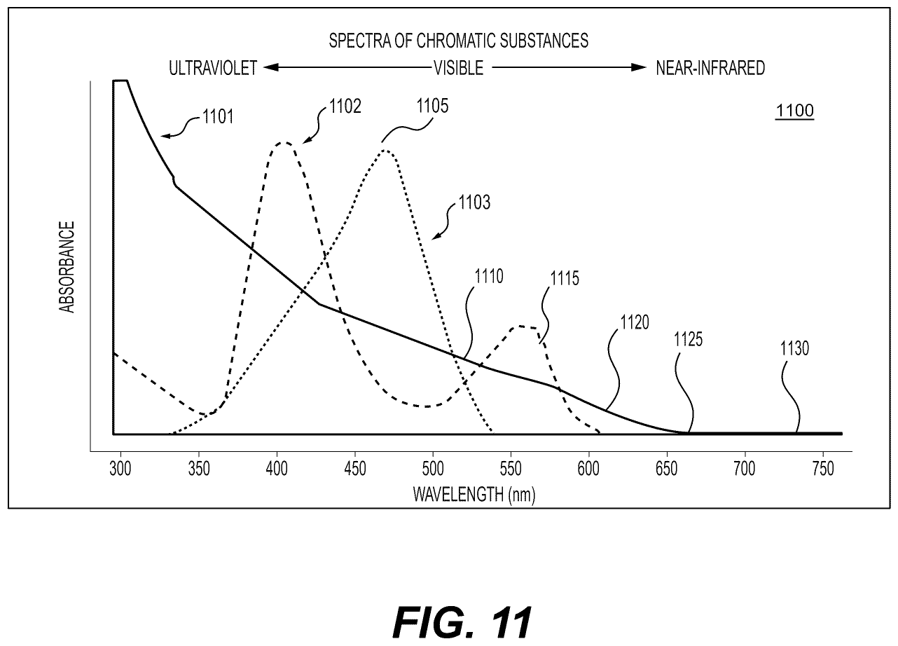

In addition to reagent-specific quality control, the system may also test plasma/serum quality for hemolysis, icterus and lipemia (HIL). In some embodiments the HIL test is performed in dedicated channel(s) of a photometry module. In other embodiments the HIL test may be performed in the ICM sample cup. In other embodiments the HIL test may be performed in a separate module. The HIL module will be described later with reference to FIG. 10 and FIG. 11.

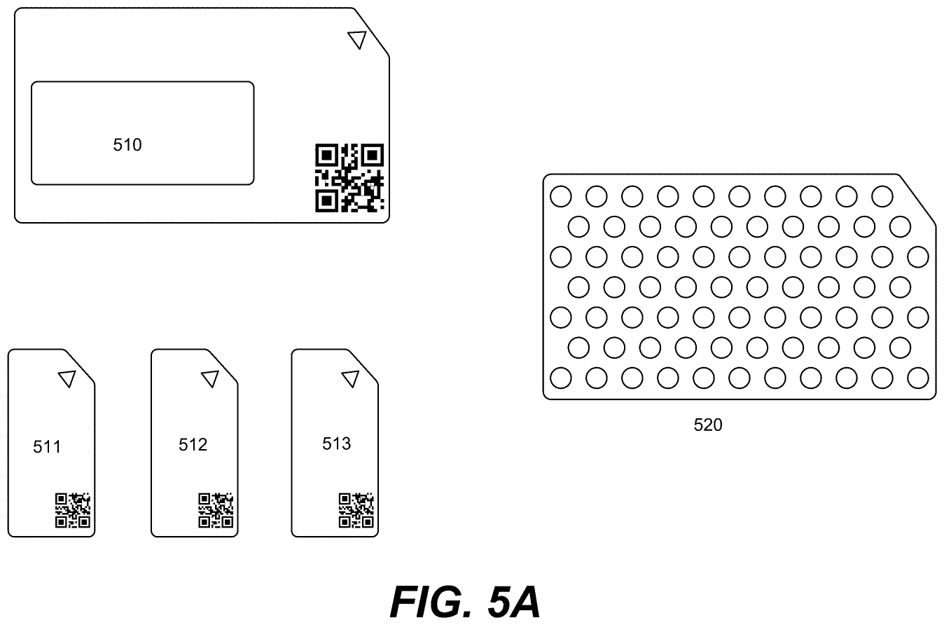

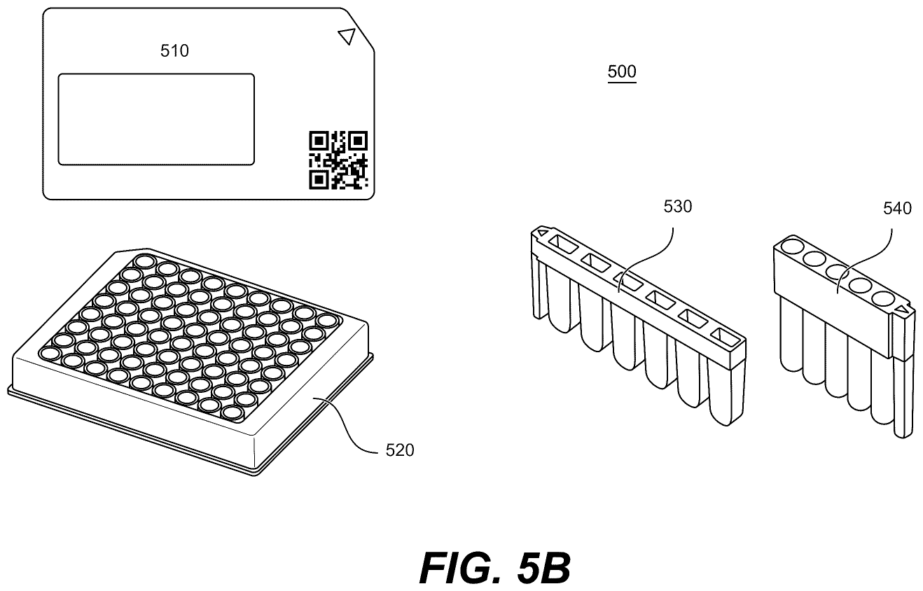

FIGS. 5A-5B depict consumables used by the integrated multimodal analytical system of FIGS. 3 and 4, according to one or more embodiments. As shown in FIGS. 5A-5B, the multimodal analytical system further comprises one or more consumable reagent plates. The reagent plate(s) hold the reagents that may be used in the assay(s). When the reagents are used up, the consumable reagent plate is replaced.

In particular, FIG. 5A depicts a cartridge 510 that resembles a well-plate. The cartridge 510 is shown with a foil seal, and cartridge 520 with the seal removed. Cartridge 510 contains all the reagents needed for clinical chemistry and hematology assays. Cartridge 510 may be a cartridge like cartridge 511, cartridge 512, and cartridge 513. Cartridge 520 may be a cartridge like cartridge 510 with the seal removed. A multimodal analytical system described herein may include one or more consumables (for example, cartridge 510) with one or more reagents for the hematology module and the chemistry module. The one or more consumables (for example, cartridge 510) may include a reagent consumable plate with a removable foil seal. As shown in FIG. 5A, the multimodal analytical system comprising test consumables includes a simple reagent package resembling a well-plate that can be manufactured with low cost, high-throughput methods (e.g. injection molding, thermoforming). This allows for small and large format menus, which can be combined to generate the information required.

FIG. 5B depicts a reagent consumable plate for the integrated multimodal analytical system, according to one or more embodiments. Cartridge 510 and cartridge 520 may be similar reagent cartridges for clinical chemistry and hematology. The system may include IAM reaction vessels 530 and IAM reagent vessels 540. FIG. 5B shows the consumables used by the analytical module, which include a strip of IAM reaction vessels 530, and a strip of IAM reagent vessels 540. In some embodiments, the multimodal system does not use reagent packs designed to perform 10's to 100's of tests each. Instead, the multimodal analytical system of the present disclosure uses a single-use cartridge.

In some embodiments the multimodal analytical system may use bulk fluids for system-level tasks (priming, washing, cleaning), but may use simple single-use consumables to hold reagents for individual assays. In some embodiments the division of fluids into this dichotomy enables the instrument to minimize the cost and complexity of the consumables while maintaining high-quality results. Because washing and cleaning fluids are available in bulk, they may be used to clean and re-use parts of the system that are typically disposed of in other point-of-care systems. For instance, the multimodal analytical system may make use of fixed probe(s) instead of disposable pipette tips, and the modules within the analytical system may make use of re-usable containers instead of disposable ones. These reusable items may be cleaned and washed with bulk fluids as required to ensure instrument performance is maintained. Cleaning and re-using hardware reduces the cost and complexity of the consumables which keeps operational costs to a minimum, while also reducing the environmental footprint of the instrument.

In some embodiments the consumable includes a plurality of wells that may be used to store the reagents needed for a plurality of assays. In some embodiments the wells in the consumable are substantially-identical to each other and can be used to hold reagents for any or almost-any of the various assays performed on the instrument. Thus, the consumable may provide great flexibility for altering the type, location, and volume of the reagents used in tests. In some embodiments the consumable has some empty wells available for menu expansion and in this context, adding a new assay to the menu is as simple as adding an additional reagent to an available well in the consumable.

In some embodiments the pipettor may perform some sample-processing steps on the consumable, but it may also aspirate reagents from the consumable and transfer them to containers in the modules (e.g. cuvettes, cups, reaction vessels) for further processing. Regardless of where the sample processing is performed, each assay substantially maintains its independence. Thus, each assay may have a unique number of reagents, sample volume, incubation period, mixing time, and read-period, and changing these process steps for one assay does not impact any other. To put it slightly differently: reagents and sample for each assay may be individually aspirated from their wells on the consumable(s), and dispensed in their proper locations. Thus, for each assay, the system may maintain direct, independent control of assay parameters including reagent volume, sample volume, and mixing parameters, along with incubation and reading times.

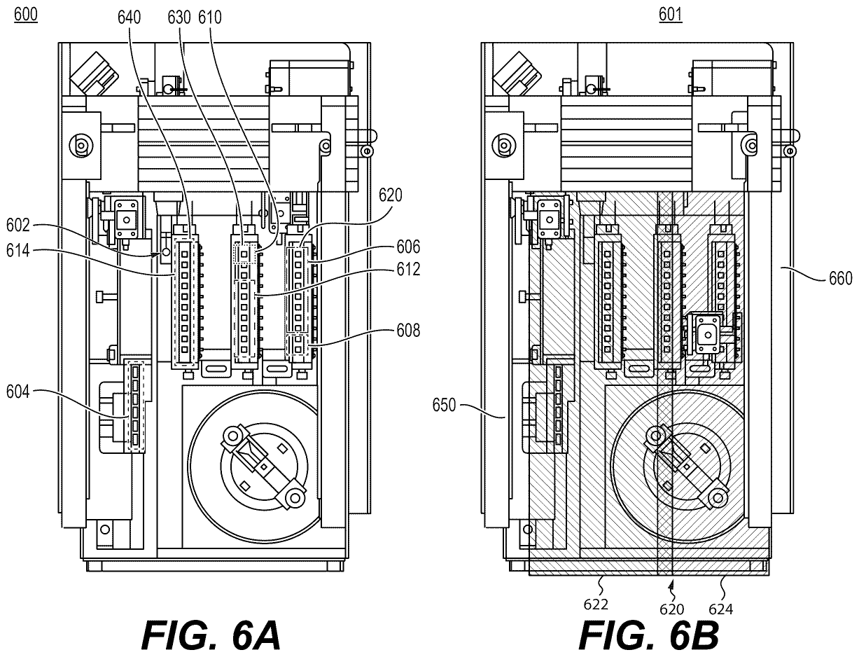

FIG. 6A depicts a top cross-sectional view of an exemplary layout of the modules of the integrated multimodal analytical system, according to one or more embodiments. As shown in FIG. 6A, system 600 may include ICM sample cup 602, IAM reaction vessels 604, and three IPMs, IPM 620, IPM 630, and IPM 640, each containing 10 cuvettes. Clinical chemistry cuvettes 606, clinical chemistry cuvettes 608, clinical chemistry cuvettes 610, clinical chemistry cuvettes 612, and clinical chemistry cuvettes 614 may be distributed among the three IPMs. In some embodiments each cuvette within an IPM is dedicated to a particular clinical chemistry test. In some embodiments the cuvette chosen for each chemistry test may be selected to make best use of system resources and analysis time. For instance, IPM 620 located on the right side of the instrument may include cuvettes 608 for running lipid assays and cuvettes 606 for a running basic metabolic panel (BMP) assays. Similarly, IPM 630 located in the center of the instrument may comprise cuvettes for seven additional assays including a hemoglobin assay and six assays beyond the BMP that comprise a comprehensive metabolic panel (CMP). Finally, IPM 640, located on the left side of the instrument may comprise cuvettes for ten other assays which are less commonly ordered.

IPM2 also has a dedicated cuvette for measuring the interfering substances Hemolysis, Icterus, and Lipemia which will be later described with reference to FIG. 10 and FIG. 11.

FIG. 6B depicts hatching over fly-zones for two working arms of the integrated multimodal analytical system, the hatching demonstrating low overlap and/or interference between those two fly-zones, according to one or more embodiments. Specifically, as shown in FIG. 6B, system 601 may include a first arm fly-zone 622, a second arm fly-zone 624, and fly-zone overlap 621. In one embodiment, fly-zone overlap 621 is minimal or even non-existent. System 601 may include the assays that run on the IPMs which may be deliberately chosen to optimize system performance. For example, the fly zones and hardware layout may be chosen to reduce the time required for assays in all three modalities to be completed. FIG. 6B illustrates the fly-zones that enable the two arms to complete a workflow that includes a comprehensive metabolic panel (CMP), a hemoglobin assay, an HIL assay, a complete blood count (CBC) with 5-part differential, and an immunoassay in a short amount of time. The left arm 650 handles all pipetting operations for the CBC, the immunoassay, the hemoglobin assay, and six comprehensive metabolic panel assays. The right arm 660 handles sample preparation (by accessing the centrifuge), performs the HIL analysis, and is further responsible for the basic metabolic panel (BMP) and lipid assays.

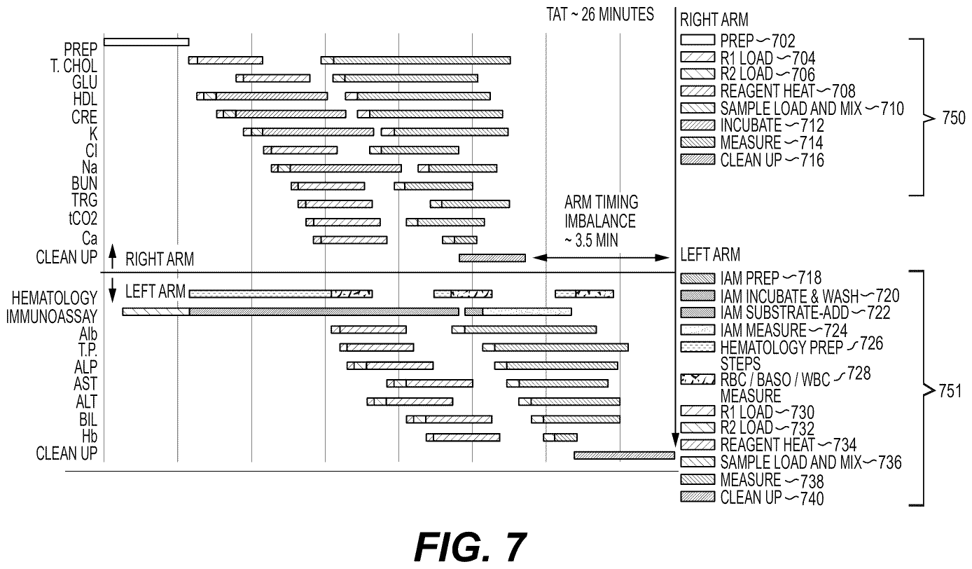

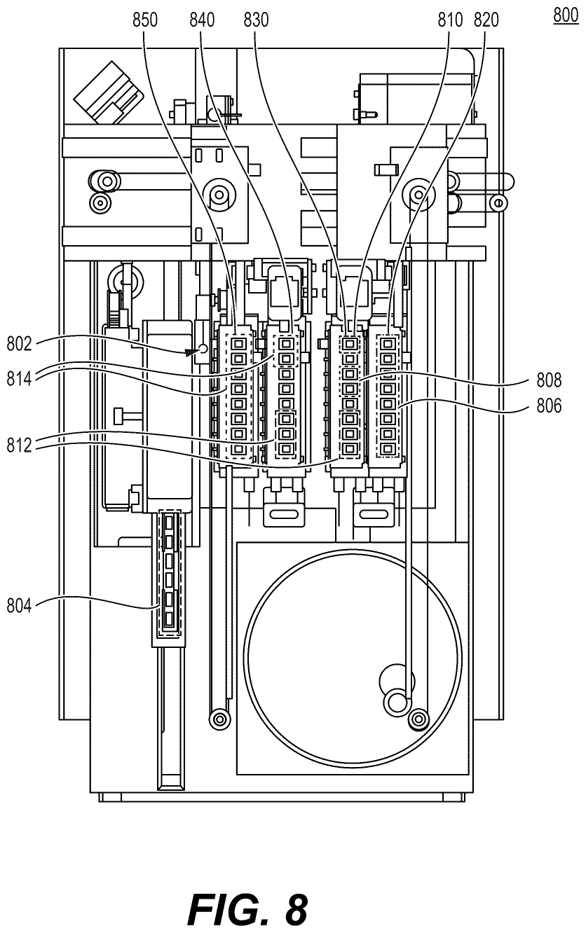

FIG. 7 depicts a timing diagram for two working arms of the integrated multimodal analytical system, according to the hardware arrangement depicted in FIG. 6A-6B, demonstrating high simultaneous productivity of those two working arms. Graph 700 may include the timing and usage of the first arm and second arm of system 600 or system 601, for example. Graph 700 may include the timing and usage for a left arm and right arm respectively. Graph 700 may include right arm operations 750 and left arm operations 751. Right arm operations 750 may include preparation 702, reagent1 (R1) load 704, reagent2 (R2) load 706, reagent heat 708, sample load and mix 710, incubate 712, measure 714, and clean up 716. Left arm operations 751 may include IAM preparation 718, IAM incubate and wash 720, IAM substrate-add 722, IAM measure 724, hematology preparation steps 726, red-blood-cell/basophil/white-blood-cell measures 728, R1 load 730, R2 load 732, reagent heat 734, sample load and mix 736, measure 738, and clean up 740. The turn-around time (TAT) may be approximately 26 minutes. The arm timing imbalance (defined as the time when one arm is finished with its work while the other is still working) may be approximately 3.5 minutes.

In particular, FIG. 7 depicts a timing diagram illustrating the process steps involved in a trimodal run. For clinical chemistry these include preparation of sample by centrifugation (“prep”), reagent loading (“RI load”, “R2 load”) sample loading, incubations, measurements, and clean-ups. For hematology, this includes sample preparation, and measurements of red blood cells (RBCs), basophils (BASO), and white blood cells (WBCs). For immunoassays this includes sample preparation, along with incubation, wash, substrate-addition, and measure steps.

Each step requires a certain amount of time to complete, and the assays are ordered so that they can be completed as quickly as possible. The location of the hardware and the order in which the assays are done can impact the total tum-around time (“TAT”), which is the total amount of time required to conduct all the assays and clean the system in preparation for its next use.

In the representative system shown in FIG. 6, the order of the assays as shown in FIG. 7 enables all the assays to be completed with a turnaround time of ˜26 minutes. The system hardware layout and the assays that run on them go hand-in-hand. The two are determined together to ensure that the two arms have minimal interaction with each other. For instance, in the example above, the right arm only needs to access the middle IPM 630 to do the HIL assay (which it does as part of the “prep”) step, while the left arm uses the middle IPM for six CMP assays and hemoglobin. The right arm does the HIL analysis before the left arm needs to access the middle IPM, and so the two arms stay out of each other's way.

In some embodiments the order in which the assays are performed is determined by a desire to minimize the turn-around-time or time-to-results. In some embodiments this may be achieved by ensuring that assays with long heat, incubation, or measure steps get started first, and by ensuring that both pipettors are operating at nearly fully duty cycle. As can be observed from inspection of FIG. 7, a full duty cycle is not always achieved for both arms. As seen in the figure the left and right arm are not active for the same amount of time. In the example provided, the right arm finishes its work ˜3.5 minutes before the left arm. This difference is the “arm timing imbalance” and represents an opportunity for efficiency improvement.

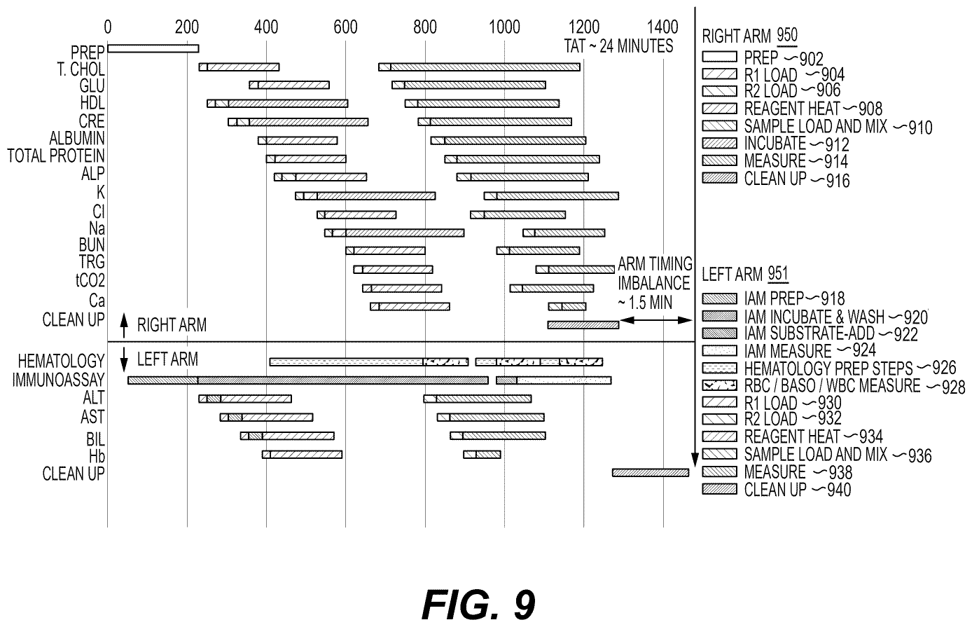

There can be alternative embodiments of the hardware and locations where the assays can be performed. Uniquely, the modularity of our system enables us to reposition hardware and shift assays around (both spatially and temporally) so as to tune and improve aspects of instrument performance. In some embodiments, the hardware may be altered and/or repositioned and the assay order changed to minimize the turnaround time. For instance, by using QTY (4) IPMs, it is possible to reconfigure the assays shown above so that the right arm takes additional clinical chemistry assays away from the left arm. This reduces the arm timing inefficiency to 1.5 minutes and results in an improved TAT time of −24 minutes. This is illustrated in FIG. 8 and FIG. 9.

FIG. 8 depicts a top cross-sectional view of another exemplary layout of the modules of the integrated multimodal analytical system, according to one or more embodiments. Specifically, as shown in FIG. 8, system 800 may include an ICM sample cup 802, IAM reaction vessels 804, and four IPMs, IPM 820, IPM 830, IPM 840, and IPM 850 containing BMP cuvettes 806, lipid cuvettes 808, an HIL cuvette 810, cuvettes for a hemoglobin and an additional six CMP assays 812, and cuvettes for additional clinical chemistry assays 814. System 800 may include the location of QTY (4) IPMs (each containing 8 cuvettes), and the location of the ICM sample cup and IAM wells.

FIG. 9 depicts a timing diagram for two working arms of the integrated multimodal analytical system, according to the hardware arrangement depicted in FIG. 8. Graph 900 may include the timing and usage of the first arm and second arm of system 800, for example. Graph 900 may include the timing and usage for a left arm and right arm respectively. Graph 900 may include right arm operations 950 and left arm operations 951. Right arm operations 950 may include preparation 902, R1 load 904, R2 load 906, reagent heat 908, sample load and mix 910, incubate 912, measure 914, and clean up 916. Left arm operations 951 may include IAM preparation 918, IAM incubate and wash 920, IAM substrate-add 922, IAM measure 924, hematology preparation steps 926, red-blood-cell/basophil/white-blood-cell measures 928, R1 load 930, R2 load 932, reagent heat 934, sample load and mix 936, measure 938, and clean up 940. The turn-around-time (TAT) may be approximately 24 minutes. Arm timing imbalance may be approximately 1.5 minutes.

In some embodiments, the integrated multimodal analytical system of FIGS. 3 and 4 has a dedicated photometry channel for performing HIL (hemolysis, icterus and lipemia) interferent analysis.

FIG. 10 depicts a custom Hemolysis, Icterus, and Lipemia (HIL) circuit board attached to a photometric module of the integrated multimodal analytical system, according to one or more embodiments. Specifically, as shown in FIG. 10A, system 1000 may include an HIL board 1010 attached to a photometric module, where the HIL board measures free hemoglobin, lipemia (lipids), and bilirubin. HIL board 1010 may include light-emitting diode 1022, light-emitting diode 1024, light-emitting diode 1026, and light-emitting diode 1028.

In some embodiments, the HIL board includes LEDs that emit two, three, four, five, or six different wavelengths of light. In some embodiments the HIL board includes surface mount LEDs. In some embodiments some of the surface mount LEDs are dual-wavelength LEDs. FIG. 10 shows an HIL board with four LEDs, two of which are dual-wavelength. The HIL board thus has the ability to interrogate the sample in the adjacent cuvette with up to six wavelengths of light. The LEDs may be chosen so that their emission wavelengths span an absorption range from 465 nm to 740 nm. This span includes a peak for bilirubin (which causes icterus) at 465 nm, and a peak for hemoglobin (which is a measure of hemolysis) at 574 nm. It also includes the 610 nm and 660 nm wavelengths where the only interferent is expected to be lipemia. It further includes the 740 nm wavelength, where little absorption from any interferent is expected.

The HIL board, in some embodiments, is used to generate a look-up table correlating H, I, and L interferent concentrations with specific combinations of absorption values observed at each wavelength. In operation, then, the HIL cuvette is filled with a diluted sample, and the absorption of the sample at each of the desired wavelengths is measured. Those absorption values are matched on the look-up table with the concentrations of H, I, and L interferents, and so the quality of the sample is assessed.

In some embodiments, the multimodal analytical system can assess the quality of the sample in an automated fashion, prior to running any of the chemistry assays, and decisions can be made in real-time about which assays to alter or flag as a result.

FIG. 11 depicts an absorption spectrum for the H, I, and L interferents, with the LED wavelengths called-out, according to one or more embodiments. Graph 1100 includes an absorption spectrum for the H, I, and L interferents. Solid line 1101 is a plot of absorption vs. wavelength for lipemic interferents. Dashed line 1102 is a plot of absorption vs. wavelength for the free hemoglobin interferent. Dotted line 1103 is a plot of absorption vs. wavelength for the bilirubin interferent. Graph 1100 also includes exemplary LED wavelengths called-out, according to one or more embodiments. Graph 1100 includes point 1105, point 1110, point 1115, point 1120, point 1125, and point 1130. Point 1105 is at 465 nm, which corresponds, or nearly-corresponds, to a peak in bilirubin absorbance. Point 1110 is at 515 nm. Point 1115 is at 574 nm, which is near a hemoglobin absorption peak. Point 1120 is at 610 nm and point 1125 is at 660 nm. Both of these wavelengths are primarily impacted by lipemic interferents. Finally, point 1130 is at 740 nm, where little or no absorption due to interfering substances is expected. The HIL board's multi-wavelength photometric approach enables the system to discriminate among different types of sample interferents. In particular, the use of LEDs emitting at 465 nm, 515 nm, 574 nm, 610 nm, 660 nm, and 740 nm allows for precise identification of unique absorption characteristics associated with bilirubin (icterus), hemoglobin (hemolysis), and lipid-induced turbidity (lipemia). This targeted selection of wavelengths provides a robust method for real-time, automated quality assessment of sample integrity by mapping the LED emission wavelengths to the corresponding absorption features for the interferents.

FIG. 10 supports the functional integration of the HIL board within the photometric module. This integration enables the multimodal analyzer to flag and optionally compensate for sample quality issues, thereby improving reliability and accuracy of downstream clinical chemistry measurements performed on the same instrument.

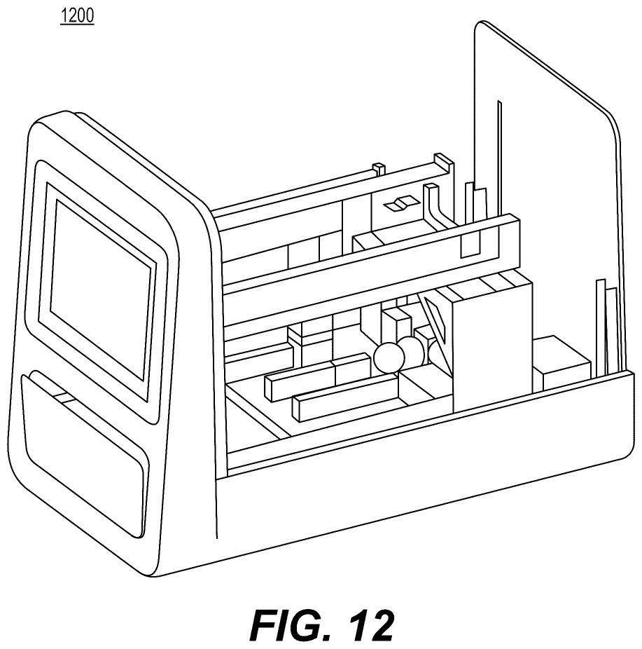

FIG. 12 provides an orthographic interior view of the integrated multimodal analytical system, according to one or more embodiments.

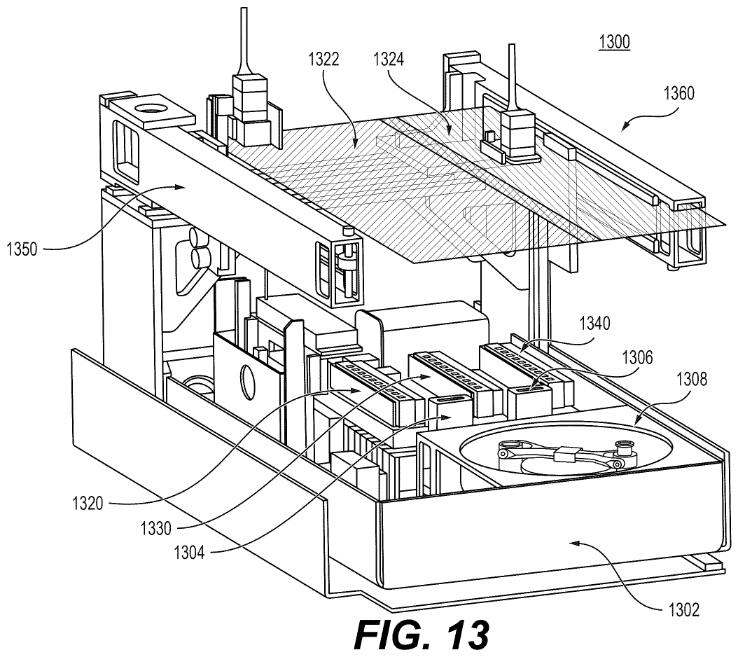

FIG. 13 provides a perspective view of an interior layout of the integrated multimodal analytical system, according to one or more embodiments. System 1300 may be a system like system 1200. System 1300 may be a multimodal analytical system. System 1300 may include housing 1302, waste/wash station 1304, waste/wash station 1306, centrifuge 1308, IPM 1320, arm one fly zone 1322, arm two fly zone 1324, IPM 1330, IPM 1340, arm one 1350, and arm two 1360.

In some embodiments, the multimodal analytical system further comprises a mixing apparatus. A non-limiting example of a mixing apparatus is a stir-pipettor in which the pipette is mounted on a flange that is eccentrically attached to a motor shaft. When the motor spins, the movement of the end of the flange where the pipette is mounted describes an oval or circular arc. The circular motion of the pipette can be used to efficiently stir liquids in fluid containers. An example of a stir-pipettor is provided in FIG. 14A-14B.

Specifically, FIG. 14A depicts an isometric view of a stir-pipettor of the integrated multimodal analytical system, according to one or more embodiments. FIG. 14B depicts a bottom view of the stirpipettor with a zoomed-in view of the pipette as it turns, according to one or more embodiments. FIG. 14B may describe a circular arc. System 1400 includes motor 1405, coupler with eccentric hole 1410, mounting bracket 1415, flange 1420, and pipette 1425. Coupler with eccentric hole 1410 may be a coupler having an eccentric-shaped hole or aperture connected to the motor 1405.

In some embodiments the movement of the pipette tip when the motor is actuated describes a rough circle having a diameter of 1 mm, 2 mm, 3 mm, 4 mm, or 5 mm. In some embodiments, the motor operates at 5, 10, 15, 20, 25, 30, 35, or 40 revolutions per minute, causing the pipette to also move in a circular arc at these frequencies.

Other stirring embodiments include magnetic stir bars or magnetic balls that may be placed inside fluid containers and actuated with a magnet, such as a bar magnet, or electromagnets that may be positioned outside of the fluid container. Movement of the bar magnet, or actuation of the electromagnets affects movement of the balls or bar magnets within the fluid container, thereby creating turbulence that mixes the fluids. Another non-limiting example of a mixing apparatus includes mechanical features such as propellors or impellors. These may reside permanently in the cuvettes or move into the fluid container to mix and retract from the cuvettes during measurement operations. In some embodiments, the multimodal analytical system comprises mechanical means of mixing one or more fluids within fluid container(s). In some embodiments, the system comprises non-mechanical means of mixing fluids within fluid container(s).

As described above, in some embodiments, the system further comprises one or more waste and wash stations.

FIG. 15 depicts an exemplary waste/wash station of the fluidic system, according to one or more embodiments. In some embodiments, the multimodal analytical system further comprises one or more waste/wash stations, each of which is also connected to a pump, as shown in FIG. 15. Specifically, as shown in FIG. 15, system 1500 includes cleaning fluid reservoir 1505, cleaning pump 1510, waste output 1515, extra-cleaning fluid hole 1520, small waste reservoir 1525, deep cleaning hole 1530, shallow cleaning hole 1535, cleaning fluid input 1540, waste tank 1570, pump 1560, valve 1550, and fluid flow 1571.

The waste/wash station(s) can be used to accept waste from the pipettor(s) and to wash the inside and outside of the pipettors. Each station may also be used as a reservoir of cleaning fluid for the pipettor to aspirate and distribute to other vessels in the system. An exemplary waste/wash station that may be used with the system is shown in FIG. 15. It includes a shallow blind hole that can be used to clean a relatively short portion of the end of the pipette. It also includes a deep blind hole that can be used to clean a longer portion of the pipette. The waste/wash station also includes a pipette cleaning fluid input that communicates through a fluidic channel to a pipette cleaning fluid hole. The waste/wash station further includes a small waste reservoir, which may be filled by spill-over from liquids exiting the cleaning fluid hole, shallow cleaning hole and/or deep cleaning hole. The waste may be removed to waste tank 1570 through waste output hole 1515 gated by valve 1550, using waste pump 1560.

During a typical cleaning operation, the pipette may first optionally eject its contents into the small waste reservoir and then move to the blind shallow cleaning hole and prime system fluid into it. This priming bathes the inside and outside of the pipette with system fluid and is often sufficient to clean both the inside and outside of the pipette.

If a more stringent clean is required, the pipette may position itself in the “extra-cleaning fluid hole”. A cleaning-fluid pump then pumps cleaning fluid into the extra-cleaning fluid hole, where it bathes the outside of the pipettor, and may be pulled into the inside of the pipettor if desired. Next, the pipettor moves to the deep cleaning hole, and there performs a series of system-fluid primes to remove residual cleaning fluid from the pipette.

In some embodiments, the waste/wash station is connected to a waste pump 1560 through a valve 1550, such that when the valve is open, the pump is fluidically connected to the waste/wash station. In some embodiments, the system further comprises a waste output and a waste tank 1570. An example of such a system is shown in FIG. 15. In some embodiments the waste pump and waste reservoir are shared by many modules in the multimodal analytical instrument, each gated by a valve.

In some embodiments, the system further comprises a cleaning fluid tank. In some embodiments, the fluidic system further comprises a cleaning pump. An example of a cleaning pump is shown in FIG. 15.

As described above, in some embodiments, the multimodal analytical system comprises one or more pipettor(s) for pipetting and/or mixing the sample and/or reagents in the cuvette(s). In some embodiments, the pipettor(s) are automated pipettor(s). In some embodiments, the system comprises a plurality of pipettors. In some embodiments, the pipettor(s) are mounted to arms on a robot, such as an XYZ gantry robot to enable them to move to different places in the system.

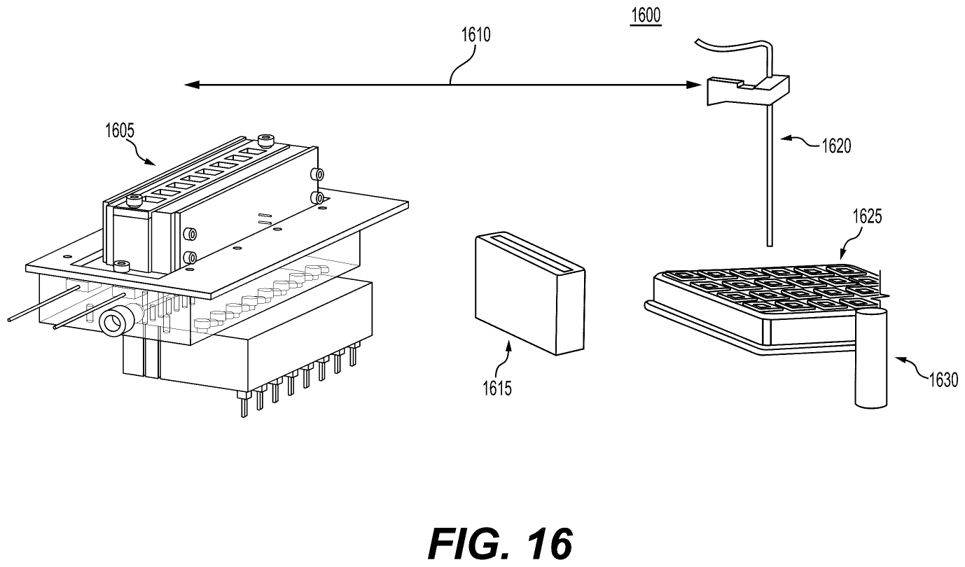

FIG. 16 depicts a pipettor configuration and orientation in association with an integrated photometry module (IPM), a waste station, and a sample holder of the integrated multimodal analytical system, according to one or more embodiments. System 1600 may include IPM 1605, waste wash station 1615, pipettor 1620, consumable well plate 1625, and serum tube 1630. Pipettor 1620 may be mounted to a robot and move in direction 1610, which may include movement in a X-Y plane, an X-Y-Z volume, and/may include angular movement.

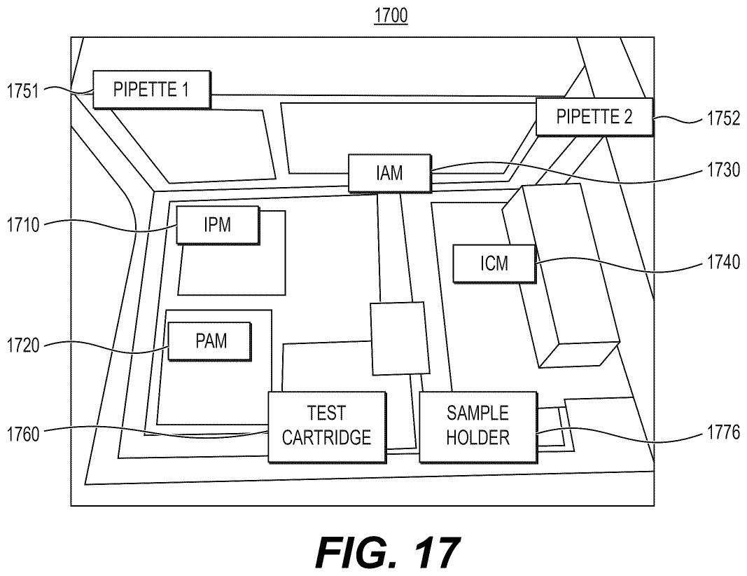

FIG. 17 depicts a conceptual schematic of the various modules of the integrated multimodal analytical system, according to one or more embodiments. Specifically, as shown in FIG. 17, system 1700 may be an integrated multimodal analytical system comprising a pipette 1751, pipette 1752, IPM 1710, PAM 1720, IAM 1730, ICM 1740, sample holder 1776, and test cartridge 1760. IPM 1710 may be a photometry module capable of performing photometric clinical chemistry assays. IAM 1730 may be an immunoassay module, capable of performing immunoassays such as magnetic-bead-based chemiluminescent immunoassays. ICM 1740 may be a flow cytometry module, capable of performing a complete blood count with five-part differential. PAM 1720 may be a pre-analytical module such as a centrifuge.

Aspects of the present disclosure include methods of using a multimodal analytical system for analysis of biological fluids for in vitro diagnostics (e.g., human and animal). In some embodiments, the biological fluid sample is selected from one or more of: whole blood, plasma, cerebral spinal fluid, urine, and other biological materials that have been substantially dissolved into a liquid.

Aspects of the present disclosure include methods of using a multimodal analytical system for environmental contamination testing. Such testing can include, but is not limited to: water testing and other environmental liquid testing such as waste materials and the like.

Aspects of the present disclosure include methods of using a multimodal analytical system for food testing, such as, but not limited to: testing of livestock for illness including infectious diseases, and testing of food products for bacterial contamination.

Aspects of the present disclosure include methods of using a multimodal analytical system for manufacturing and product quality control, such as testing biological therapeutic products including cell cultures and media, or testing of animals used to measure efficacy and toxicity of therapeutic products, or testing of other liquid substances related to manufacturing and product quality (e.g., nutrient supplements).

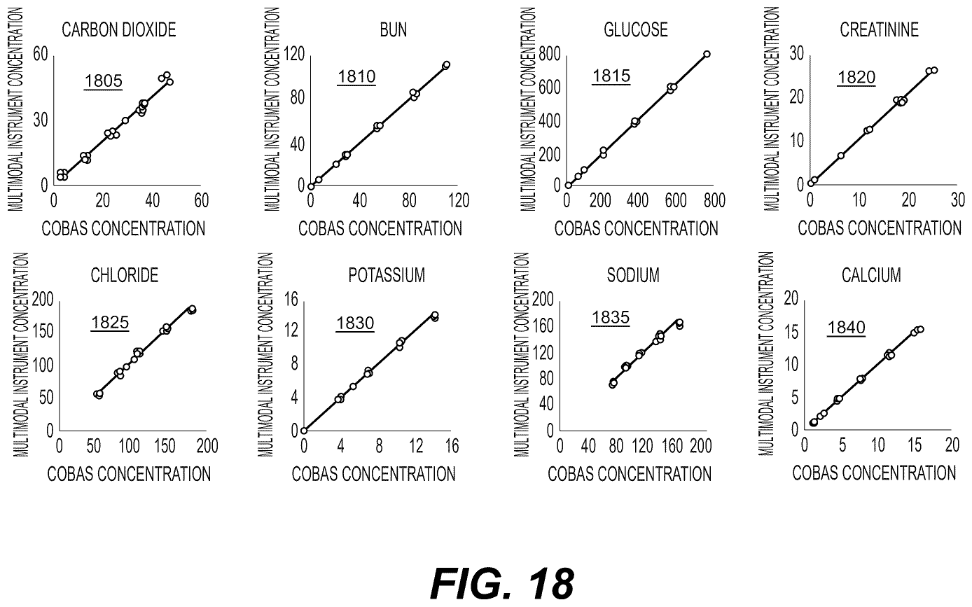

The multimodal analytical system of the present disclosure was used to perform analysis on various samples on different analytical modules within the system. Examples of output parameters measured by the multimodal system are provided in FIGS. 18-20.

Specifically, FIG. 18 depicts linearity curves for a Basic Metabolic Panel (BMP) measured from one or more modules (e.g., IPM) of the multimodal analytical system, according to one or more embodiments. The graphs show the concentration of analytes measured by the multimodal instrument that is the subject of this disclosure on the Y-axis, versus the concentration of analytes measured by a predicate (Roche Diagnostics Cobas) clinical chemistry analyzer. Graph 1805 may include performance data from carbon dioxide. Graph 1810 may include performance data from blood urea nitrogen (BUN). Graph 1815 may include performance data from glucose. Graph 1820 may include performance data from creatinine. Graph 1825 may include performance data from chloride. Graph 1830 may include performance data from potassium. Graph 1835 may include performance data from sodium. Graph 1840 may include performance data from calcium.

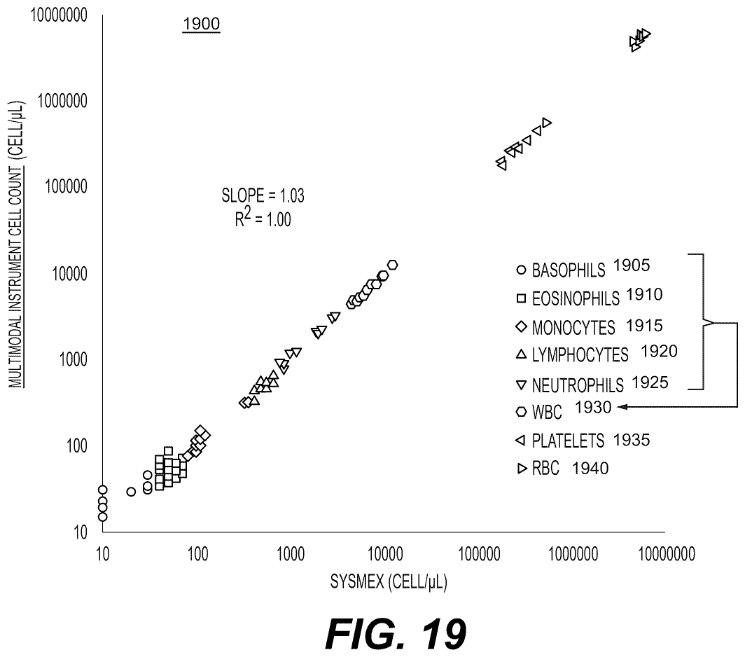

FIG. 19 depicts linearity curves for a Complete Blood Count (CBC) measured from one or more modules (e.g., ICM) of the multimodal analytical system, according to one or more embodiments. The graph shows the cell counts per microliter measured by the multimodal instrument that is the subject of this disclosure on the Y-axis, versus the cell counts per microliter measured by a predicate (Sysmex) hematology analyzer. Graph 1900 may include basophils 1905, eosinophils 1910, monocytes 1915, lymphocytes 1920, neutrophils 1925, total white blood cells, (WBC) representing the sum of the basophil, eosinophil, monocyte, lymphocyte, and neutrophil counts 1930, platelets 1935, and red blood cells (RBC) 1940. The systems described herein, for example system 1200 or system 1300, may include a system to perform one or more complete blood counts. The system to perform one or more complete blood counts may be a flow cytometer, for example.

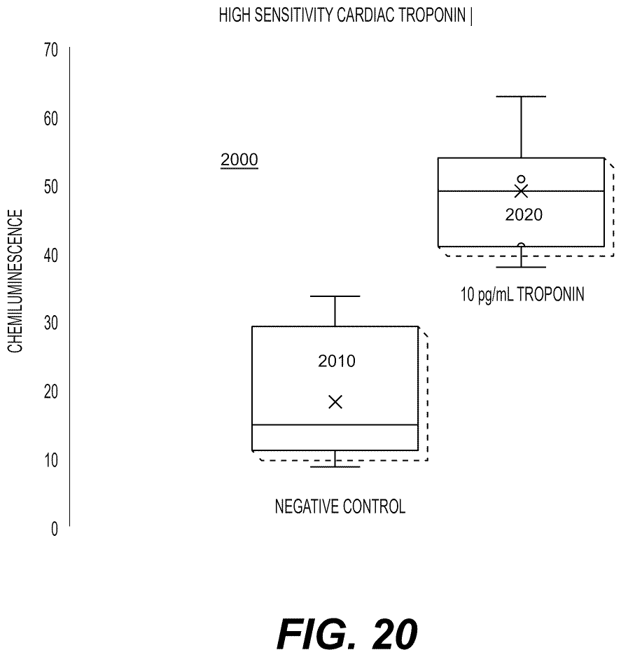

FIG. 20 shows Immunoassay (Troponin hsTni) performance data measured from one or more modules (e.g., integrated immunoassay module IAM) of the multimodal analytical system, according to one or more embodiments. Graph 2000 may include data charting chemiluminescence signal arising from tests using negative control 2010 and samples spiked with 10 pg/mL of troponin I 2020, illustrating the high sensitivity of the assay..

The data in FIG. 18, FIG. 19, and FIG. 20, may be produced by the different modules on the multimodal analytical system from a single sample, and illustrate that the instrument can generate multiple data streams representing multiple analytical techniques. In some embodiments, the multimodal analytical system may capture data from its more than one modules and integrate the data streams to provide a more comprehensive snapshot of health than any one modality can provide.

There are a variety of disease states for which a single-modality instrument, such as a chemistry, hematology, or immuno-analyzer is insufficient for a firm diagnosis. For instance, anemia can be caused by a variety of pathologies. If the hematology module (ICM) measures low red blood cell counts (indicating anemia), tests from other modalities may help elucidate the root cause. If the clinical chemistry module (IPM) concurrently reveals that liver enzymes AST and ALT are elevated, it suggests inflammation or damage to the liver, whereas if the liver enzymes are normal while blood urea nitrogen and creatinine are elevated, a provider might suspect kidney problems. Yet again, if the anemia is concurrent with elevated potassium levels, the cause may be due to tissue injury, certain medications, or poorly-controlled diabetes. The results of a fructosamine or a1c chemistry test from the IPM may rule in or out the latter diagnosis, while tissue injury is often associated with elevated creatine kinase.

Data from the multimodal analytical system may be organized and reported in ways that help the healthcare practitioner analyze the data. For instance, results from different modalities that indicate a particular disease state may be automatically flagged or grouped together. Quantitative correlations between modalities may be reported and may help the practitioner determine a diagnosis from multiple candidates.

In addition, the multimodal system may use data from one or more data-streams to suggest additional testing, and may even be configured to automatically reflex to certain additional tests on the basis of the results it obtains. For instance, anemia coupled with normal liver enzyme levels, but low total cholesterol and sodium levels suggests the possibility of a thyroid problem and might trigger the multimodal analytical instrument to automatically run a thyroid stimulating hormone (TSH) immunoassay. In this way, an integrated system becomes more than the sum of its parts.

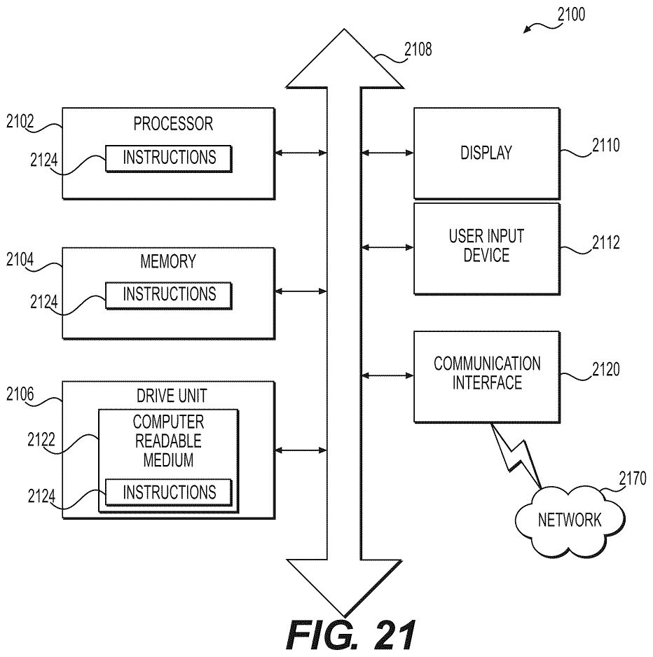

FIG. 21 depicts a schematic diagram of a computing bus and processing controller of the integrated multimodal analytical system, according to one or more embodiments. FIG. 21 depicts an exemplary system infrastructure for a controller, according to one or more embodiments. Controller 2100 may include one or more controllers. As seen in the figures, controller 2100 may be a controller as seen in multimodal analytical instrument 300. The controller 2100 may include a set of instructions that can be executed to cause the controller 2100 to perform any one or more of the methods or computer based functions disclosed herein. The controller 2100 may operate as a standalone device or may be connected, for example, using a network, to other computer systems or peripheral devices. System 1300 may include a controller 2100. System 1200 may include a controller 2100.

In a networked deployment, the controller 2100 may operate in the capacity of a server or as a client in a server-client user network environment, or as a peer computer system in a peer-to-peer (or distributed) network environment. The controller 2100 can also be implemented as or incorporated into various devices, such as a power converter, a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the controller 2100 can be implemented using electronic devices that provide voice, video, or data communication. Further, while the controller 2100 is illustrated as a single system, the term “system” shall also be taken to include any collection of systems or sub-systems that individually or jointly execute a set, or multiple sets, of instructions to perform one or more computer functions.

As depicted in FIG. 21, the controller 2100 may include a processor 2102, e.g., a central processing unit (CPU), a graphics processing unit (GPU), or both. The processor 2102 may be a component in a variety of systems. The processor 2102 may be one or more general processors, digital signal processors, application specific integrated circuits, field programmable gate arrays, servers, networks, digital circuits, analog circuits, combinations thereof, or other now known or later developed devices for analyzing and processing data. The processor 2102 may implement a software program, such as code generated manually (i.e., programmed).

The controller 2100 may include a memory 2104 that can communicate via a bus 2108. The memory 2104 may be a main memory, a static memory, or a dynamic memory. The memory 2104 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 2104 includes a cache or random-access memory for the processor 2102. In alternative implementations, the memory 2104 is separate from the processor 2102, such as a cache memory of a processor, the system memory, or other memory. The memory 2104 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 2104 is operable to store instructions executable by the processor 2102. The functions, acts or tasks illustrated in the figures or described herein may be performed by the processor 2102 executing the instructions stored in the memory 2104. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As depicted, the controller 2100 may further include a display 2110, such as a liquid crystal display (LCD), an organic light emitting diode (OLED), a flat panel display, a solid-state display, a cathode ray tube (CRT), a projector, a printer or other now known or later developed display device for outputting determined information. The display 2110 may act as an interface for the user to see the functioning of the processor 2102, or specifically as an interface with the software stored in the memory 2104 or in the drive unit 2106.

Additionally or alternatively, the controller 2100 may include an input device 2112 configured to allow a user to interact with any of the components of controller 2100. The input device 2112 may be a number pad, a keyboard, or a cursor control device, such as a mouse, or a joystick, touch screen display, remote control, or any other device operative to interact with the controller 2100.

The controller 2100 may also or alternatively include drive unit 2106 implemented as a disk or optical drive. The drive unit 2106 may include a computer-readable medium 2122 in which one or more sets of instructions 2124, e.g. software, can be embedded. Further, the instructions 2124 may embody one or more of the methods or logic as described herein. The instructions 2124 may reside completely or partially within the memory 2104 and/or within the processor 2102 during execution by the controller 2100. The memory 2104 and the processor 2102 also may include computer-readable media as discussed above.

In some systems, a computer-readable medium 2122 includes instructions 2124 or receives and executes instructions 2124 responsive to a propagated signal so that a device connected to a network 2170 can communicate voice, video, audio, images, or any other data over the network 2170. Further, the instructions 2124 may be transmitted or received over the network 2170 via a communication port or interface 2120, and/or using a bus 2108. The communication port or interface 2120 may be a part of the processor 2102 or may be a separate component. The communication port or interface 2120 may be created in software or may be a physical connection in hardware. The communication port or interface 2120 may be configured to connect with a network 2170, external media, the display 2110, or any other components in controller 2100, or combinations thereof. The connection with the network 2170 may be a physical connection, such as a wired Ethernet connection or may be established wirelessly as discussed below. Likewise, the additional connections with other components of the controller 2100 may be physical connections or may be established wirelessly. The network 2170 may alternatively be directly connected to a bus 2108.

While the computer-readable medium 2122 is depicted to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 2122 may be non-transitory, and may be tangible.

The computer-readable medium 2122 can include a solid-state memory such as a memory card or other package that houses one or more non-volatile read-only memories. The computer-readable medium 2122 can be a random-access memory or other volatile re-writable memory. Additionally or alternatively, the computer-readable medium 2122 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The controller 2100 may be connected to a network 2170. The network 2170 may define one or more networks including wired or wireless networks. The wireless network may be a cellular telephone network, an 802.11, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 2170 may include wide area networks (WAN), such as the Internet, local area networks (LAN), campus area networks, metropolitan area networks, a direct connection such as through a Universal Serial Bus (USB) port, or any other networks that may allow for data communication. The network 2170 may be configured to couple one computing device to another computing device to enable communication of data between the devices. The network 2170 may generally be enabled to employ any form of machine-readable media for communicating information from one device to another. The network 2170 may include communication methods by which information may travel between computing devices. The network 2170 may be divided into sub-networks. The sub-networks may allow access to all of the other components connected thereto or the sub-networks may restrict access between the components. The network 2170 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component or object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the operations of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

Thus, as described above throughout, the present disclosure provides a multimodal analytical system that adapts multiple technologies used by large laboratories and seamlessly integrates them into a single, compact and easy-to-operate analyzer. One or more embodiments may provide a method of integrating these technologies to reduce user interaction, improve ease-of-use, improve test quality, reduce wait times, and reduce the cost of testing. Furthermore the methods may enable a broad test menu, and may enable the instrument to synergistically analyze data from multiple modalities.

One or more embodiments may provide a multimodal analytical system having a modular architecture that leverages commonalities between the modalities to make efficient use of system-wide resources including bulk wash and cleaning fluids, along with fluid, motion, control, and software systems, while still enabling the use of simple, low-cost, single-use consumables that house the reagents needed for each assay. In some embodiments the consumables may be kept simple in-part because the system hardware and much of the hardware within the modules is re-usable and can be washed with the wash and cleaning fluids. The simplicity of the consumables enables each assay to maintain substantially-independent processing steps, and provides a flexibility that accommodates changes to the instrument and/or testing menu.

While the invention has been particularly shown and described with reference to a preferred embodiment and various alternate embodiments, it will be understood by persons skilled in the relevant art that various changes in form and details can be made therein without departing from the spirit and scope of the invention. For instance, the embodiments described herein have been shown and described with particular modules that include a plasma-preparation module, photometry module, cytometry module, and immunoassay module, and examples of each have been provided. However different modules could equally-well be used in the system. For instance an imaging module and/or a Coulter module could be used in addition-to or in-lieu-of the cytometry module for cellular assays. A module to detect DNA or RNA in samples could also be integrated into the system.

The system-level hardware could also be altered without fundamentally altering the key innovative aspects of this invention. For instance, as described herein, the system level hardware includes pipettors mounted to robotic arm(s) that move fluids from one place to another. However, an alternative embodiment for the system-hardware might use an array of syringes, valves, and tubes to aspirate reagents from the simple consumable cartridge and dispense them to desired locations within their modules. Other system-level architectures are also possible.

All references, issued patents and patent applications cited within the body of the instant specification are hereby incorporated by reference in their entirety, for all purposes.

What is claimed is:

1. An analytical system for analyzing fluids, the system comprising:
a fluid sample staging module disposed within a housing and configured to stage fluid samples for one or more analyses;
one or more analytical modules disposed within the housing and comprising one or more of:
a cytometry analysis module configured to perform particulate counts;
a chemistry analysis module configured to perform chemistry assays; and
an immunoassay analysis module configured to perform immunoassay testing;
common fluid handling hardware disposed in the housing and configured to convey fluid samples or portions thereof between the fluid sample staging module, the one or more analytical modules, and a consumable reagent cartridge inserted into the housing, the consumable reagent cartridge comprising an array of wells accessible to the fluid handling hardware, the array of wells containing reagents for a first set of tests or controls by the one or more analytical modules; and
common waste fluid and wash fluid handling hardware disposed in the housing and in fluid communication with the one or more analytical modules and the common fluid handling hardware, for cleaning the common fluid handling hardware and fluid-contacting surfaces of the one or more analytical modules before interaction with a second consumable reagent cartridge containing reagents for a second set of tests or controls by the one or more analytical modules.

2. The analytical system of claim 1, wherein the common waste fluid and wash fluid handling hardware is configured to clean the fluid handling hardware and fluid-contacting hardware of the one or more analytical modules between each set of tests by the one or more analytical modules for reuse of the fluid handling hardware with second and subsequent consumable reagent cartridges, each of the second and subsequent consumable reagent cartridges containing only enough reagents to operate respective second and subsequent set of tests or controls by the one or more analytical modules.

3. The analytical system of claim 1, wherein the one or more analytical modules disposed within the housing comprise two or more of:
a cytometry analysis module configured to perform complete blood count analysis;
a chemistry analysis module configured to perform clinical chemistry assays; and
an immunoassay analysis module configured to perform immunoassay testing.

4. The analytical system of claim 1, wherein the fluid sample staging module is a plasma-preparation module configured to separate plasma or serum from whole blood samples.

5. The analytical system of claim 1, wherein the one or more analytical modules comprise a hematology analysis module configured to perform a complete blood count with 5-part differential, and a chemistry analysis module configured to perform one or more multiplexed panels of clinical chemistry assays, including a comprehensive metabolic panel (CMP).

6. The analytical system of claim 1, wherein the one or more analytical modules comprise a hematology analysis module that uses dual-angle Mie-scattering to measure the mean corpuscular hemoglobin concentration (MCHC), mean corpuscular volume (MCV), and mean platelet volume (MPV).

7. The analytical system of claim 1, wherein the one or more analytical modules comprise a cytometry analysis module comprising a flow cytometer.

8. The analytical system of claim 1, wherein the one or more analytical modules comprise a chemistry analysis module comprising a photometric module that determines the concentration of analytes through fluorescence and/or absorbance measurements through a fluid sample.

9. The analytical system of claim 1, wherein the one or more analytical modules comprise an immunoassay analysis module that performs magnetic-bead based chemiluminescent tests.

10. The analytical system of claim 1, wherein the common fluid handling hardware comprises one or more probes configured to aspirate and transfer fluids and/or reagents from the consumable reagent cartridge to the one or more analytical modules.

11. The analytical system of claim 1, wherein the common fluid handling hardware comprises at least two automated pipettors configured to operate independently, each of the two automated pipettors being mounted to a separate movable arm and/or gantry robot configured to move in two or three dimensions.

12. The analytical system of claim 1, further comprising:
a control system disposed within the housing and configured to control the common fluid handling hardware and common waste fluid and wash fluid handling hardware to enable simultaneously or nearly simultaneously analyzing a single fluid sample using two more of the analytical modules.

13. The analytical system of claim 1, wherein the fluid handling hardware comprises one or more independently controllable pipette probes configured to:
aspirate liquids from the consumable reagent cartridge; and
transfer the aspirated liquids to vessels within the one or more analytical modules.

14. The analytical system of claim 1, wherein the consumable reagent cartridge:
is made from a monolithic piece of molded or thermoformed plastic; and
has one or more wells filled with one or more reagents useable by the one or more analytical modules; and
wherein each well in the consumable reagent cartridge is independent and fluidically isolated from every other well in the consumable reagent cartridge, and
wherein the consumable reagent cartridge is sealed with a pierceable foil seal covering its array of wells, making the array of wells and reagents therein accessible to the fluid handling hardware.

15. A modular, point-of-care diagnostic system for analyzing blood, the system comprising:
a blood sample preparation module disposed within a housing and configured to separate plasma or serum from whole blood samples;
one or more analytical modules disposed within the housing and comprising one or more of:
a hematology analysis module configured to perform particulate counts and perform a complete blood count analysis; and
a chemistry analysis module configured to perform chemistry assays;
common biological fluid handling hardware disposed in the housing and configured to convey blood samples or portions thereof between the blood sample preparation module, the one or more analytical modules, and a consumable reagent cartridge inserted into the housing, the consumable reagent cartridge comprising one or more reagents for a first set of tests or controls, the one or more reagents being accessible to the common biological fluid handling hardware for transfer to the one or more analytical modules; and
common wash fluid handling hardware disposed in fluid communication with the one or more analytical modules and the common biological fluid handling hardware for preparing the common fluid handling hardware and fluid-contacting surfaces of the one or more analytical modules for interaction with a second consumable reagent cartridge containing reagents for a second set of tests or controls by the one or more analytical modules.

16. A modular, point-of-care diagnostic system for analyzing blood, the system comprising:
a blood sample preparation module disposed within a housing and configured to separate plasma or serum from whole blood samples; and
two analytical modules disposed within the housing, comprising:
a hematology analysis module configured to perform particulate counts and perform a complete blood count analysis; and
a chemistry analysis module configured to perform chemistry assays.

17. A modular analytical system for analyzing fluids, the system comprising:
a fluid sample staging module disposed within a housing and configured to stage fluid samples for one or more analyses;
two or more analytical modules disposed within the housing and comprising one or more of:
a cytometry analysis module configured to perform particulate counts;
a chemistry analysis module configured to perform chemistry assays; and
an immunoassay analysis module configured to perform immunoassay testing;
common fluid handling hardware disposed in the housing and configured to convey fluid samples or portions thereof between the fluid sample staging module, the one or more analytical modules, and a consumable reagent cartridge inserted into the housing, the consumable reagent cartridge comprising an array of wells accessible to the fluid handling hardware;
common waste fluid and wash fluid handling hardware disposed in the housing and in communication with the two or more analytical modules and the common fluid handling hardware; and
a controller configured to control the common fluid handling hardware to simultaneously interact with the two or more analytical modules, to generate a diagnostic report of a fluid sample based on outputs received from the two or more analytical modules, and to control the common wash fluid handling hardware to clean the common fluid handling hardware before analysis of a second fluid sample.

18. The modular analytical system of claim 17, wherein the controller is further configured to control the common fluid handling hardware to convey portions of a fluid sample from the fluid sample staging module to wells of the consumable reagent cartridge, and to simultaneously convey fluid from two or more wells of the consumable reagent cartridge to vessels of the two or more analytical modules.

19. The modular analytical system of claim 17, wherein the controller is further configured to control the common waste fluid and wash fluid handling hardware to clean the common fluid handling hardware and fluid-contacting surfaces of the two or more analytical modules between analyses of second and subsequent fluid samples using second and subsequent consumable reagent cartridges.

20. The modular analytical system of claim 17, wherein the controller is further configured to:
use outputs from the two or more analytical modules to identify correlations between the outputs, organize the outputs relative to a particular diagnosis or set of diagnoses, or initiate or recommend additional analysis based on the outputs; and
control the movement of fluid between the fluid sample staging module, wells of the consumable reagent cartridge, vessels of the two or more analytical modules to minimize time to produce the outputs from the two or more analytical modules.
